(19) United States
(12) Patent Application Publication
(10) Pub. No.: US 2024/0173374 A1
(43) Pub. Date: May 30, 2024

(54) LIPOSOMAL FORMULATIONS OF BORONIC ACID CONTAINING ACTIVE AGENTS

(71) Applicant: Ohio State Innovation Foundation, Columbus, OH (US)

(72) Inventor: Robert LEE, Lewis Center, OH (US)

(21) Appl. No.: 18/279,004

(22) PCT Filed: Feb. 25, 2022
(86) PCT No.: PCT/US2022/017911

§ 371 Date: Aug. 25, 2023

Related U.S. Application Data
(60) Provisional application No. 63/153,947, filed on Feb. 25, 2021.

(51) Int. Cl. A61K 38/05; A61K 9/127; A61P 35/00

(52) U.S. Cl. CPC A61K 38/05; A61K 9/1271; A61P 35/00

(57) ABSTRACT

Described are liposomal formulation for the improved delivery of boronic acid esters of boronic acid therapeutic agents. The liposomal formulation can include (i) liposomes formed from a vesicle-forming lipid; and (ii) a capturing agent-active agent complex encapsulated in the liposomes.

### CROSS-REFERENCE TO RELATED APPLICATIONS

The application claims the benefit of U.S. Provisional Application No. 63/153,947, filed Feb. 25, 2021, which is hereby incorporated herein by reference in its entirety.

### BACKGROUND

Bortezomib (PS-341, Velcade, BTZ) is a dipeptide boronic acid and inhibitor of proteasome. BTZ was developed by Millennium (now part of Takeda) and is currently approved for relapsed multiple myeloma (MM) and Mantle cell lymphoma (MCL). The clinical formulation of BTZ (Velcade) is 3.5 mg per vial with 35 mg mannitol as complexing agent. BTZ has serious dose-liming side effects, primarily peripheral neuropathy and myelosuppression, which limit its clinical application. In a clinical trial for systemic lupus erythematosus (SLE), 7/12 dropped out due to side effects despite efficacy. In an AML trial, neurotoxicity led to 4/7 to drop out. Preclinical study showed that BTZ cannot reach effective dose in AML murine model due to toxicity. In theory, BTZ is likely effective against a number of tumors, including both hematologic malignancies and solid tumors. There is great potential for expanded clinical utility for BTZ if its formulation can be improved that reduces its side effects. Several liposomal formulations of BTZ have been developed previously. However, these formulations lack sufficient stability or are based on new excipients which are not readily available or have not previously been used in man, which limit their potential for clinical translation. For example, BTZ has been loaded into liposomes via pH gradient using sorbitol, meglumine, or amino-lactose as encapsulated complexing agents. These agents form complexes with BTZ at high pH, which affects formulation stability. In another study, BTZ was complexed to a lipophilic catechol derivative for liposomal incorporation. This is problematic due to the need for a new complexing agent with unknown safety profile and relatively fast release of a lipophilic drug complex from the liposomes. Therefore, there is a need for new L-BTZ liposomal formulations that reduce the side effects associated with the current clinical formulations of BTZ.

The compositions and methods disclosed herein address these and other needs.

### SUMMARY

Provided herein are liposomal formulations and methods of making and using the formulations. In some embodiments, the liposomal formulation includes: (i) liposomes formed from a vesicle-forming lipid; and (ii) a capturing agent-active agent complex encapsulated in the liposomes.

In some embodiments, the capturing agent-active agent complex is a compound defined by Formula A:

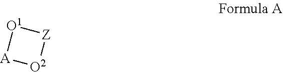

or a pharmaceutically acceptable salt thereof,
wherein:
A represents an active agent comprising a boronic acid, and
Z together with O¹ and O² represent a capture agent, wherein Z comprises an aromatic diol substituted one or more charged moieties,
wherein A is bound to the capture agent so as to form a boronic ester having the structure below

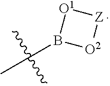

In some embodiments, the capturing agent-active agent complex is a compound defined by Formula A-1:

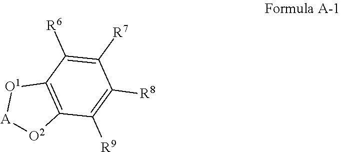

or a pharmaceutically acceptable salt thereof,
wherein:
A represents an active agent comprising a boronic acid, and
R⁶-R⁹ are independently hydrogen, oxo, hydroxy, carboxylic acid, carboxylate, sulfonic acid, sulfonate, phosphoryl group, a primary amine, a secondary amine, a tertiary amine, quaternary amine, substituted or unsubstituted alkyl group, cycloalkyl group, heterocyclyl group, aryl group, heteroaryl group, alkylaryl group, arylalkyl group, alkylcycloalkyl group, alkylheterocyclyl group, alkylheteroaryl group, alkoxy group, alkylthio group, or alkylamino group; and at least one of R⁶-R⁹ is charged moiety;
wherein A is bound to O¹ and O² so as to form a boronic ester having the structure below

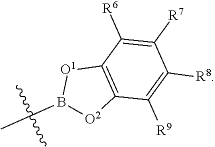

In some embodiments, the capturing agent-active agent complex is a compound defined by Formula A-2:

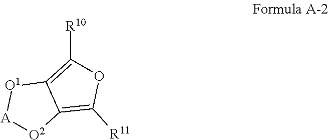

or a pharmaceutically acceptable salt thereof,
wherein:
A represents an active agent comprising a boronic acid, and
R¹⁰ and R¹¹ are independently hydrogen, oxo, hydroxy, carboxylic acid, carboxylate, sulfonic acid, sulfonate, phosphoryl group, a primary amine, a secondary amine, a tertiary amine, quaternary amine, substituted or unsubstituted alkyl group, cycloalkyl group, heterocyclyl group, aryl group, heteroaryl group, alkylaryl group, arylalkyl group, alkylcycloalkyl group, alkylheterocyclyl group, alkylheteroaryl group, alkoxy group, alkylthio group, or alkylamino group; and at least one of R¹⁰ or R¹¹ is an charged moiety;
wherein A is bound to O¹ and O² so as to form a boronic ester having the structure below

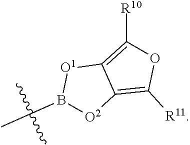

In some embodiments, the capturing agent-active agent complex is a compound defined by Formula A-3:

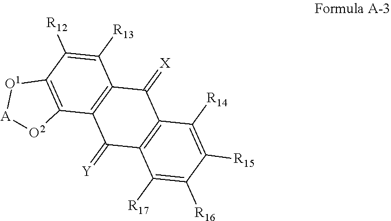

or a pharmaceutically acceptable salt thereof,
wherein:
A represents an active agent comprising a boronic acid, and
R¹²-R¹⁷ are independently hydrogen, oxo, hydroxy, carboxylic acid, carboxylate, sulfonic acid, sulfonate, phosphoryl group, a primary amine, a secondary amine, a tertiary amine, quaternary amine, substituted or unsubstituted alkyl group, cycloalkyl group, heterocyclyl group, aryl group, heteroaryl group, alkylaryl group, arylalkyl group, alkylcycloalkyl group, alkylheterocyclyl group, alkylheteroaryl group, alkoxy group, alkylthio group, or alkylamino group; and at least one of R¹²-R¹⁷ is a charged moiety;
wherein A is bound to O¹ and O² so as to form a boronic ester having the structure below

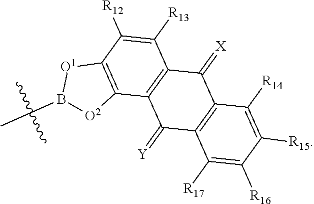

In some embodiments, the capturing agent-active agent complex is a compound defined by Formula I:

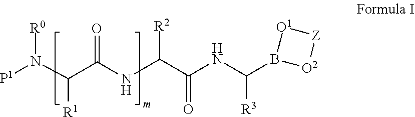

wherein
P¹ is hydrogen or an amino-group protecting moiety;
R⁰ is hydrogen or an alkyl group;
R¹, R², and R³ are independently hydrogen, an alkyl group, a cycloalkyl group, a heterocyclyl group, an aryl group, a heteroaryl group, an alkoxy, or —CH₂—R⁴;
R⁴ is an aryl group, an alkylaryl group, an arylalkyl group, a cycloalkyl group, an alkylcycloalkyl group, a heterocyclyl group, an alkylheterocyclyl group, a heteroaryl group, an alkylheteroaryl group, an alkoxy group, or an alkylthio group;
m is 0, 1, or 2;
Z together with O¹ and O² represent a capture agent, wherein Z comprises an aromatic diol substituted one or more charged moieties,
a pharmaceutically acceptable salt thereof.

In some embodiments, the one or more charged moieties can comprise an anionic moiety (e.g., from one to three anionic moieties). In some embodiments, the one or more charged moieties can comprise a cationic moiety (e.g., from one to three cationic moieties). In some embodiments, the one or more charged moieties can comprise one or more anionic moieties and one or more cationic moieties.

In some embodiments, the capture agent is a diol. In some embodiments, the capture agent is an aromatic diol. In some embodiments, the aromatic diol is a catechol or a furan diol.

In some embodiments, the catechol is tiron or a derivative thereof, alizarin red S or a derivative thereof, L,D-3,4-dihydroxyphenylalanine, or a catecholamine such as dopamine, epinephrine, norepinephrine, or a derivative thereof. In some embodiments, the furan diol is ascorbic acid or a derivative.

In some embodiments, the capturing agent-active agent complex is a compound defined by Formula IA:

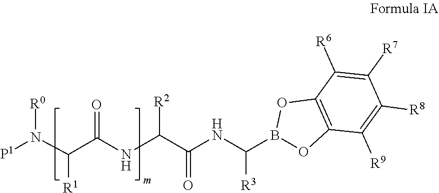

wherein
P¹ is hydrogen or an amino-group protecting moiety;
R⁰ is hydrogen or an alkyl group;
R¹, R², and R³ are independently hydrogen, an alkyl group, a cycloalkyl group, a heterocyclyl group, an aryl group, a heteroaryl group, an alkoxy,or —CH₂—R⁴;
R⁴ is an aryl group, an alkylaryl group, an arylalkyl group, a cycloalkyl group, an alkylcycloalkyl group, a heterocyclyl group, an alkylheterocyclyl group, a heteroaryl group, an alkylheteroaryl group, an alkoxy group, or an alkylthio group;
m is 0, 1, or 2;
R⁶-R⁹ are independently hydrogen, oxo, hydroxy, carboxylic acid, carboxylate, sulfonic acid, sulfonate, phosphoryl group, a primary amine, a secondary amine, a tertiary amine, quaternary amine, substituted or unsubstituted alkyl group, cycloalkyl group, heterocyclyl group, aryl group, heteroaryl group, alkylaryl group, arylalkyl group, alkylcycloalkyl group, alkylheterocyclyl group, alkylheteroaryl group, alkoxy group, alkylthio group, or alkylamino group; and at least one of R⁶-R⁹ is a charged moiety; or
a pharmaceutically acceptable salt thereof.

In some embodiments, each charged moiety can comprise an anionic moiety. In some embodiments, each charged moiety can comprise a cationic moiety. In some embodiments, at least one of R⁶-R⁹ can comprise an anionic moiety and at least one of R⁶-R⁹ can comprise a cationic moiety.

In some embodiments, the capturing agent-active agent complex is a compound defined by Formula IB:

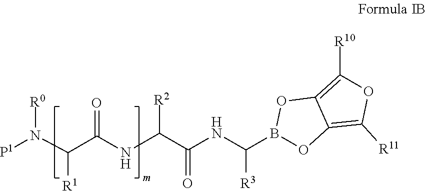

wherein
P¹ is hydrogen or an amino-group protecting moiety;
R⁰ is hydrogen or an alkyl group;
R¹, R², and R³ are independently hydrogen, an alkyl group, a cycloalkyl group, a heterocyclyl group, an aryl group, a heteroaryl group, an alkoxy, or —CH₂—R⁴;
R⁴ is an aryl group, an alkylaryl group, an arylalkyl group, a cycloalkyl group, an alkylcycloalkyl group, a heterocyclyl group, an alkylheterocyclyl group, a heteroaryl group, an alkylheteroaryl group, an alkoxy group, or an alkylthio group;
m is 0, 1, or 2;
R¹⁰ and R¹¹ are independently hydrogen, oxo, hydroxy, carboxylic acid, carboxylate, sulfonic acid, sulfonate, phosphoryl group, a primary amine, a secondary amine, a tertiary amine, quaternary amine, substituted or unsubstituted alkyl group, cycloalkyl group, heterocyclyl group, aryl group, heteroaryl group, alkylaryl group, arylalkyl group, alkylcycloalkyl group, alkylheterocyclyl group, alkylheteroaryl group, alkoxy group, alkylthio group, or alkylamino group; and at least one of R¹⁰ or R¹¹ is a charged moiety; or
a pharmaceutically acceptable salt thereof.

In some embodiments, each charged moiety can comprise an anionic moiety. In some embodiments, each charged moiety can comprise a cationic moiety.

In some embodiments, m is 0. In some embodiments, P¹ is R⁵—C(O)—, R⁵—S(O)₂—, R⁵—NH—C(O)—, or R⁵—O—C(O)—, wherein R⁵ is an alkyl group, an aryl group, an alkylaryl group, an arylalkyl group, a cycloalkyl group, an alkylcycloalkyl group, a heterocyclyl group, an alkylheterocyclyl group, a heteroaryl group, or an alkylheteroaryl group. In some embodiments, P¹ is R⁵—C(O)—, R⁵—S(O)₂—, R⁵—NH—C(O)—, or R⁵—O—C(O)—, and R⁵ is a heteroaryl group. In some embodiments, P¹ is (2-pyrazine)carbonyl. In some embodiments, R³ is an isobutyl group.

In some embodiments, the capturing agent-active agent complex is defined by Formula IC

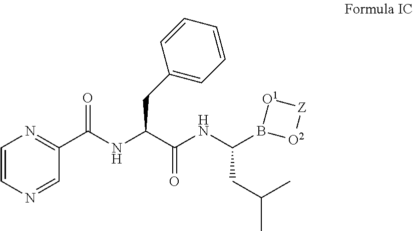

wherein
Z, together with O¹ and O², represent a capture agent, wherein Z comprises one or more charged moieties. In some embodiments, the capture agent is as previously defined for Formula I.

In some embodiments, the one or more charged moieties can comprise an anionic moiety (e.g., from one to three anionic moieties). In some embodiments, the one or more charged moieties can comprise a cationic moiety (e.g., from one to three cationic moieties). In some embodiments, the one or more charged moieties can comprise one or more anionic moieties and one or more cationic moieties.

In some embodiments, the capturing agent-active agent complex is a compound defined by Formula ID:

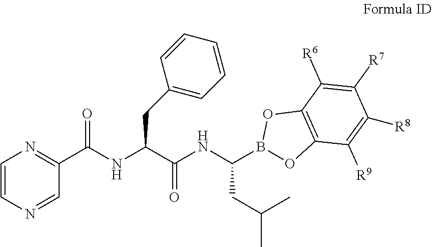

wherein
R⁶-R⁹ are independently hydrogen, oxo, hydroxy, carboxylic acid, carboxylate, sulfonic acid, sulfonate, phosphoryl group, a primary amine, a secondary amine, a tertiary amine, quaternary amine, substituted or unsubstituted alkyl group, cycloalkyl group, heterocyclyl group, aryl group, heteroaryl group, alkylaryl group, arylalkyl group, alkylcycloalkyl group, alkylheterocyclyl group, alkylheteroaryl group, alkoxy group, alkylthio group, or alkylamino group; and at least one of R⁶-R⁹ is an charged moiety; or
a pharmaceutically acceptable salt thereof.

In some embodiments, each charged moiety can comprise an anionic moiety. In some embodiments, each charged moiety can comprise a cationic moiety. In some embodiments, at least one of R⁶-R⁹ can comprise an anionic moiety and at least one of R⁶-R⁹ can comprise a cationic moiety.

In some other embodiments, the capturing agent-active agent complex is a compound defined by Formula IE:

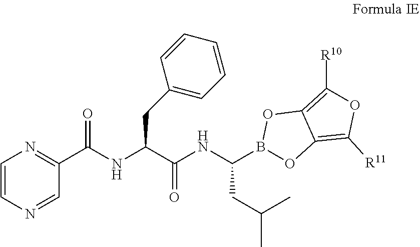

wherein
R¹⁰ and R¹¹ are independently hydrogen, oxo, hydroxy, carboxylic acid, carboxylate, sulfonic acid, sulfonate, phosphoryl group, a primary amine, a secondary amine, a tertiary amine, quaternary amine, substituted or unsubstituted alkyl group, cycloalkyl group, heterocyclyl group, aryl group, heteroaryl group, alkylaryl group, arylalkyl group, alkylcycloalkyl group, alkylheterocyclyl group, alkylheteroaryl group, alkoxy group, alkylthio group, or alkylamino group; and at least one of R¹⁰ or R¹¹ is an charged moiety; or
a pharmaceutically acceptable salt thereof.

In some embodiments, each charged moiety can comprise an anionic moiety. In some embodiments, each charged moiety can comprise a cationic moiety.

In some embodiments, the capturing agent-active agent complex is a compound defined by Formula IF:

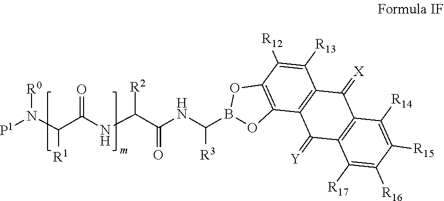

wherein
P¹ is hydrogen or an amino-group protecting moiety;
R⁰ is hydrogen or an alkyl group;
R¹, R², and R³ are independently hydrogen, an alkyl group, a cycloalkyl group, a heterocyclyl group, an aryl group, a heteroaryl group, an alkoxy, or —CH₂—R⁴;
m is 0, 1, or 2;
X and Y are independently N, O, or S;
R¹²-R¹⁷ are independently hydrogen, oxo, hydroxy, carboxylic acid, carboxylate, sulfonic acid, sulfonate, phosphoryl group, a primary amine, a secondary amine, a tertiary amine, quaternary amine, substituted or unsubstituted alkyl group, cycloalkyl group, heterocyclyl group, aryl group, heteroaryl group, alkylaryl group, arylalkyl group, alkylcycloalkyl group, alkylheterocyclyl group, alkylheteroaryl group, alkoxy group, alkylthio group, or alkylamino group; and at least one of R¹²-R¹⁷ is a charged moiety; or
a pharmaceutically acceptable salt thereof.

In some embodiments, each charged moiety can comprise an anionic moiety. In some embodiments, each charged moiety can comprise a cationic moiety. In some embodiments, at least one of R¹²-R¹⁷ can comprise an anionic moiety and at least one of R¹²-R¹⁷ can comprise a cationic moiety.

In some embodiments, the capturing agent-active agent complex is a compound defined by Formula IG:

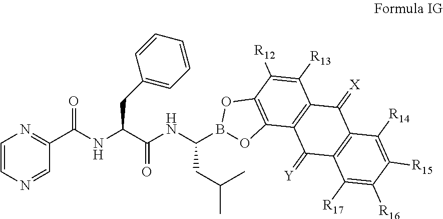

wherein
X and Y are independently N, O, or S;
R¹²-R¹⁷ are independently hydrogen, oxo, hydroxy, carboxylic acid, carboxylate, sulfonic acid, sulfonate, phosphoryl group, a primary amine, a secondary amine, a tertiary amine, quaternary amine, substituted or unsubstituted alkyl group, cycloalkyl group, heterocyclyl group, aryl group, heteroaryl group, alkylaryl group, arylalkyl group, alkylcycloalkyl group, alkylheterocyclyl group, alkylheteroaryl group, alkoxy group, alkylthio group, or alkylamino group; and at least one of R¹²-R¹⁷ is an charged moiety; or
a pharmaceutically acceptable salt thereof.

In some embodiments, each charged moiety can comprise an anionic moiety. In some embodiments, each charged moiety can comprise a cationic moiety. In some embodiments, at least one of R¹²-R¹⁷ can comprise an anionic moiety and at least one of R¹²-R¹⁷ can comprise a cationic moiety.

In some embodiments, the vesicle-forming lipid includes: a lipid, a phospholipid, and a PEG-conjugated phospholipid. In some embodiments, the one or more lipid comprises cholesterol. In some embodiments, the phospholipid comprises 1,2-distearoyl-sn-glycero-3-phosphocholine (DSPC), 1,2-dipalmitoyl-sn-glycero-3-phosphocholine (DPPC), hydrogenated soybean phosphatidylcholine (HSPC), or sphingomyelin (SPH). The phospholipid can be a negatively charged lipid such as distearoylphosphatidylglycerol (DSPG), dipalmitoylphosphatidylglycerol (DPPG), or dicetylphophosphate. In some embodiments, the PEG-conjugated phospholipid comprises N-(methylpolyoxyethylene oxycarbonyl)-1,2-distearoyl-sn-glycero-3-phosphoethanolamine (DSPE-PEG).

In some embodiments, the phospholipid, the lipid, and the PEG-phospholipid are present in a molar percent ratio ranging from about 40-100:0-60:0-20, respectively. For example, the phospholipid, the lipid, and the PEG-phospholipid are present in a molar percent ratio of about 50:25:25, about 90:5:5, or of about 60:35:5. In some embodiments, the lipid is cholesterol.

In some embodiments, the liposome has a mean particle size (diameter) ranging from 45 nm to 30 μm. For example from 50 nm to 140 nm, from 50 nm to 250 nm, or from 60 to 140 nm, such as about 50 nm, about 60 nm, about 70 nm, about 80 nm, about 90 nm, about 100 nm, about 110 nm, about 120 nm, about 130 nm, about 140 nm, about 150 nm, about 180, about 200 nm, about 220 nm, or about 250 nm, about 500 nm, about 1 μm, about 5 μm, about 10 μm, about 20 μm, or about 30 μm. In some embodiments, the phospholipid, the lipid, capture agent, and the PEG-phospholipid are present in a molar percent ratio ranging from about 50:25:20:5 to about 85:5:5:5, such as in a molar percent ratio of about 50:25:20:5 or about 60:15:20:5.

Described herein are also methods of treating a cancer in a subject in need thereof, including administering a therapeutically effective amount of a liposomal formulation described herein. In some embodiments, the cancer is selected from B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers, small cell lung cancer, non-small cell lung cancer, neuroblastoma, glioblastoma, ovarian cancer, pancreatic cancer, prostate cancer, skin cancers, melanoma, basal cell carcinoma, squamous cell carcinoma, liver cancer, squamous cell carcinomas of the mouth, throat, larynx, and lung, cervical cancer, cervical carcinoma, breast cancer, renal cancer, genitourinary cancer, pulmonary cancer, esophageal carcinoma, head and neck carcinoma, large bowel cancer, hematopoietic cancers; testicular cancer;

colon and rectal cancers, prostatic cancer, AIDS-related lymphomas, or AIDS-related sarcomas.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIG. 1 shows a reaction scheme of bortezomib binding to tiron at basic condition to form a boronic ester.

FIG. 2 shows pH-dependent partitioning of BTZ in the presence of diol compounds. BTZ was combined with 50 mM of meglumine, tiron, glucose or mannitol and % of the drug in the chloroform phase was determined by OD at 270 nm.

FIG. 3A-3C shows antitumor activity of L-BTZ in cell-derived NCI-H929 multiple myeloma murine xenograft models. (3A) anti-tumor effect, (3B) body weight loss, and (3C) survival curves result in vivo.

FIG. 4 shows BTZ plasma clearance for L-BTZ and Velcade after single dose on rats.

FIG. 5 shows a therapeutic efficacy of L-BTZ in OPM2 multiple myeloma murine xenograft model.

Like reference symbols in the various drawings indicate like elements.

### DETAILED DESCRIPTION

A number of embodiments of the disclosure have been described. Nevertheless, it will be understood that various modifications may be made without departing from the spirit and scope of the invention. Accordingly, other embodiments are within the scope of the following claims.

### Definitions

To facilitate understanding of the disclosure set forth herein, a number of terms are defined below. Unless defined otherwise, all technical and scientific terms used herein generally have the same meaning as commonly understood by one of ordinary skill in the art to which this disclosure belongs. Publications cited herein and the materials for which they are cited are specifically incorporated by reference.

### General Definitions

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing quantities of ingredients, reaction conditions, geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

As used in this specification and the following claims, the terms “comprise” (as well as forms, derivatives, or variations thereof, such as “comprising” and “comprises”) and “include” (as well as forms, derivatives, or variations thereof, such as “including” and “includes”) are inclusive (i.e., open-ended) and do not exclude additional elements or steps. For example, the terms “comprise” and/or “comprising,” when used in this specification, specify the presence of stated features, integers, steps, operations, elements, and/or components, but do not preclude the presence or addition of one or more other features, integers, steps, operations, elements, components, and/or groups thereof. Accordingly, these terms are intended to not only cover the recited element(s) or step(s), but may also include other elements or steps not expressly recited. Furthermore, as used herein, the use of the terms “a”, “an”, and “the” when used in conjunction with an element may mean “one,” but it is also consistent with the meaning of “one or more,” “at least one,” and “one or more than one.” Therefore, an element preceded by “a” or “an” does not, without more constraints, preclude the existence of additional identical elements.

The use of the term “about” applies to all numeric values, whether or not explicitly indicated. This term generally refers to a range of numbers that one of ordinary skill in the art would consider as a reasonable amount of deviation to the recited numeric values (i.e., having the equivalent function or result). For example, this term can be construed as including a deviation of ±10 percent of the given numeric value provided such a deviation does not alter the end function or result of the value. Therefore, a value of about 1% can be construed to be a range from 0.9% to 1.1%. Furthermore, a range may be construed to include the start and the end of the range. For example, a range of 10% to 20% (i.e., range of 10%-20%) can includes 10% and also includes 20%, and includes percentages in between 10% and 20%, unless explicitly stated otherwise herein.

It is understood that when combinations, subsets, groups, etc. of elements are disclosed (e.g., combinations of components in a composition, or combinations of steps in a method), that while specific reference of each of the various individual and collective combinations and permutations of these elements may not be explicitly disclosed, each is specifically contemplated and described herein.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Administration” to a subject includes any route of introducing or delivering to a subject an agent. Administration can be carried out by any suitable route, including oral, topical, intravenous, subcutaneous, transcutaneous, transdermal, intramuscular, intra joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra- articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

As used here, the terms “beneficial agent” and “active agent” are used interchangeably herein to refer to a chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition. The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Inactivate”, “inactivating” and “inactivation” means to decrease or eliminate an activity, response, condition, disease, or other biological parameter due to a chemical (covalent bond formation) between the ligand and a its biological target.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, prevention of the occurrence of symptoms and/or their underlying cause, and improvement or remediation of damage. In particular, the term “treatment” includes the alleviation, in part or in whole, of the symptoms of coronavirus infection (e.g., sore throat, blocked and/or runny nose, cough and/or elevated temperature associated with a common cold). Such treatment may include eradication, or slowing of population growth, of a microbial agent associated with inflammation.

The term “anticancer” refers to the ability to treat or control cellular proliferation and/or tumor growth at any concentration.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms. As used herein, the term “preventing” a disorder or unwanted physiological event in a subject refers specifically to the prevention of the occurrence of symptoms and/or their underlying cause, wherein the subject may or may not exhibit heightened susceptibility to the disorder or event. In particular embodiments, “prevention” includes reduction in risk of coronavirus infection in patients. However, it will be appreciated that such prevention may not be absolute, i.e., it may not prevent all such patients developing a coronavirus infection, or may only partially prevent an infection in a single individual. As such, the terms “prevention” and “prophylaxis” may be used interchangeably.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount”. However, an appropriate “effective’ amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition. Therapeutically effective and prophylactically effective amounts of a given therapeutic agent will typically vary with respect to factors such as the type and severity of the disorder or disease being treated and the age, gender, and weight of the subject. The term “therapeutically effective amount” can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

As used herein, “pharmaceutically acceptable salt” is a derivative of the disclosed compound in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH2)n—COOH where n is 0-4, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985).

Also, as used herein, the term “pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

A “control” is an alternative subject or sample used in an experiment for comparison purposes. A control can be “positive” or “negative.”

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician. Administration of the therapeutic agents can be carried out at dosages and for periods of time effective for treatment of a subject. In some embodiments, the subject is a human.

### Chemical Definitions

Terms used herein will have their customary meaning in the art unless specified otherwise. The organic moieties mentioned when defining variable positions within the general formulae described herein (e.g., the term “halogen”) are collective terms for the individual substituents encompassed by the organic moiety. The prefix Cₙ-Cₘ indicates in each case the possible number of carbon atoms in the group.

The term “alkyl,” as used herein, refers to saturated straight, branched, primary, secondary or tertiary hydrocarbons, including those having 1 to 20 atoms. In some examples, alkyl groups will include C₁-C₁₂, C₁-C₁₀, C₁-C₈, C₁-C₆, C₁-C₅, C₁-C₄, C₁-C₃, or C₁-C₂ alkyl groups. Examples of C₁-C₁₀ alkyl groups include, but are not limited to, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-l-methylpropyl, 1-ethyl-2-methylpropyl, heptyl, octyl, 2-ethylhexyl, nonyl and decyl groups, as well as their isomers. Examples of C₁-C₄-alkyl groups include, for example, methyl, ethyl, propyl, 1-methylethyl, butyl, 1-methylpropyl, 2-methylpropyl, and 1,1-dimethylethyl groups.

Cyclic alkyl groups or “cycloalkyl” groups include cycloalkyl groups having from 3 to 10 carbon atoms. Cycloalkyl groups can include a single ring, or multiple condensed rings. In some examples, cycloalkyl groups include C₃-C₄, C₄-C₇, C₅-C₇, C₄-C₆, or C₅-C₆ cyclic alkyl groups. Non-limiting examples of cycloalkyl groups include adamantyl, cyclopropyl, cyclobutyl, cyclopentyl, cyclohexyl, cycloheptyl, cyclooctyl and the like.

Alkyl and cycloalkyl groups can be unsubstituted or substituted with one or more moieties chosen from alkyl, halo, oxo (C═O), haloalkyl, hydroxyl, carboxyl, acyl, acyloxy, amino, alkyl- or dialkylamino, amido, arylamino, alkoxy, aryloxy, nitro, cyano, azido, thiol, imino, sulfonic acid, sulfonate, sulfate, sulfonyl, sulfanyl, sulfinyl, sulfamonyl, ester, phosphonyl, phosphinyl, phosphoryl, phosphine, thioester, thioether, acid halide, anhydride, oxime, hydrazine, carbamate, carboxylic acid, carboxylate, phosphoric acid, phosphate, phosphonate, or any other viable functional group that does not inhibit the biological activity of the compounds of the invention, either unprotected, or protected as necessary, as known to those skilled in the art, for example, as described in Greene, et al., Protective Groups in Organic Synthesis, John Wiley and Sons, Third Edition, 1999, hereby incorporated by reference.

Terms including the term “alkyl,” such as “alkylamino” or “dialkylamino,” will be understood to comprise an alkyl group as defined above linked to another functional group, where the group is linked to the compound through the last group listed, as understood by those of skill in the art.

The term “alkenyl,” as used herein, refers to both straight and branched carbon chains which have at least one carbon-carbon double bond. In some examples, alkenyl groups can include C₂-C₂₀ alkenyl groups. In other examples, alkenyl can include C₂-C₁₂, C₂-C₁₀, C₂-C₈, C₂-C₆, or C₂-C₄ alkenyl groups. In one embodiment of alkenyl, the number of double bonds is 1-3, in another embodiment of alkenyl, the number of double bonds is one or two. Other ranges of carbon-carbon double bonds and carbon numbers are also contemplated depending on the location of the alkenyl moiety on the molecule. “C₂-C₁₀-alkenyl” groups can include more than one double bond in the chain. The one or more unsaturations within the alkenyl group can be located at any position(s) within the carbon chain as valence permits. In some examples, when the alkenyl group is covalently bound to one or more additional moieties, the carbon atom(s) in the alkenyl group that are covalently bound to the one or more additional moieties are not part of a carbon-carbon double bond within the alkenyl group. Examples of alkenyl groups include, but are not limited to, ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl; 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3 -methyl-2-pentenyl, 4-methyl-2-pentenyl, 1 -methyl-3 -pentenyl, 2-methyl-3-pentenyl, 3 -methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3 -butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl groups.

The term “alkynyl,” as used herein, refers to both straight and branched carbon chains which have at least one carbon-carbon triple bond. In one embodiment of alkynyl, the number of triple bonds is 1-3; in another embodiment of alkynyl, the number of triple bonds is one or two. In some examples, alkynyl groups include from C₂-C₂₀ alkynyl groups. In other examples, alkynyl groups can include C₂-C₁₂, C₂-C₈, C₂-C₆ or C₂-C₄ alkynyl groups. Other ranges of carbon-carbon triple bonds and carbon numbers are also contemplated depending on the location of the alkenyl moiety on the molecule. For example, the term “C₂-C₁₀-alkynyl” as used herein refers to a straight-chain or branched unsaturated hydrocarbon group having 2 to 10 carbon atoms and containing at least one triple bond, such as ethynyl, prop-1-yn-1-yl, prop-2-yn-1-yl, n-but-1-yn-1-yl, n-but-1-yn-3-yl, n-but-1-yn-4-yl, n-but-2-yn-1-yl, n-pent-1-yn-1-yl, n-pent-1-yn-3-yl, n-pent-1-yn-4-yl, n-pent-1-yn-5-yl, n-pent-2-yn-1-yl, n-pent-2-yn-4-yl, n-pent-2-yn-5-yl, 3-methylbut-1-yn-3-yl, 3-methylbut-1-yn-4-yl, n-hex-1-yn-1-yl, n-hex-1-yn-3-yl, n-hex-1-yn-4-yl, n-hex-1-yn-5-yl, n-hex-1-yn-6-yl, n-hex-2-yn-1-yl, n-hex-2-yn-4-yl, n-hex-2-yn-5-yl, n-hex-2-yn-6-yl, n-hex-3-yn-1-yl, n-hex-3-yn-2-yl, 3-methylpent-1-yn-1-yl, 3-methylpent-1-yn-3-yl, 3-methylpent-1-yn-4-yl, 3-methylpent-1-yn-5-yl, 4-methylpent-1-yn-1-yl, 4-methylpent-2-yn-4-yl, and 4-methylpent-2-yn-5-yl groups.

Alkenyl and alkynyl groups can be unsubstituted or substituted with one or more moieties chosen from alkyl, halo, oxo (C═O), haloalkyl, hydroxyl, carboxyl, acyl, acyloxy, amino, alkyl- or dialkylamino, amido, arylamino, alkoxy, aryloxy, nitro, cyano, azido, thiol, imino, sulfonic acid, sulfonate, sulfate, sulfonyl, sulfanyl, sulfinyl, sulfamonyl, ester, phosphonyl, phosphinyl, phosphoryl, phosphine, thioester, thioether, acid halide, anhydride, oxime, hydrazine, carbamate, carboxylic acid, carboxylate, phosphoric acid, phosphate, phosphonate, or any other viable functional group that does not inhibit the biological activity of the compounds of the invention, either unprotected, or protected as necessary, as known to those skilled in the art, for example, as described in Greene, et al., Protective Groups in Organic Synthesis, John Wiley and Sons, Third Edition, 1999.

The term “aryl,” as used herein, refers to a monovalent aromatic carbocyclic group of from 6 to 14 carbon atoms. Aryl groups can include a single ring or multiple condensed rings. In some examples, aryl groups include C₆-C₁₀ aryl groups. Aryl groups include, but are not limited to, phenyl, biphenyl, naphthyl, tetrahydronaphtyl, phenylcyclopropyl and indanyl. Aryl groups can be unsubstituted or substituted by one or more moieties chosen from halo, oxo (C═O), cyano, alkoxy, aryloxy, nitro, hydroxy, mercapto, acyl, acyloxy, amino, amido, carboxylic acid, carboxylate, cyano, azido, thiol, imino, sulfonic acid, sulfonate, phosphoric acid, phosphonate, sulfate, sulfonyl, sulfanyl, sulfinyl, sulfamonyl, ester, phosphonyl, phosphinyl, phosphoryl, phosphine, thioester, thioether, acid halide, anhydride, oxime, hydrazine, carbamate, alkyl, alkenyl, alkynyl, cycloalkyl, cycloalkenyl, haloalkyl, haloalkenyl, haloalkynyl, halocycloalkyl, halocycloalkenyl, alkoxy, alkenyloxy, alkynyloxy, haloalkoxy, haloalkenyloxy, haloalkynyloxy, cycloalkoxy, cycloalkenyloxy, halocycloalkoxy, halocycloalkenyloxy, alkylthio, haloalkylthio, cycloalkylthio, halocycloalkylthio, alkylsulfinyl, alkenylsulfinyl, alkynyl-sulfinyl, haloalkylsulfinyl, haloalkenylsulfinyl, haloalkynylsulfinyl, alkyl sulfonyl, alkenyl sulfonyl, alkynylsulfonyl, haloalkyl-sulfonyl, haloalkenylsulfonyl, haloalkynylsulfonyl, alkylamino, alkenylamino, alkynylamino, di(alkyl)amino, di(alkenyl)-amino, di(alkynyl)amino, or trialkylsilyl.

The term “alkylaryl,” as used herein, refers to an aryl group that is bonded to a parent compound through a diradical alkylene bridge, (—CH₂—)ₙ, where n is 1-12 (e.g., n is from 1 to 6) and where “aryl” is as defined above. The term “arylalkyl,” as used herein, refers to an aryl group, as defined above, which is substituted by an alkyl group, as defined above.

The term “alkylcycloalkyl,” as used herein, refers to a cycloalkyl group that is bonded to a parent compound through a diradical alkylene bridge, (—CH₂—)ₙ, where n is 1-12 (e.g., n is from 1 to 6) and where “cycloalkyl” is as defined above.

The term “alkoxy,” as used herein, refers to alkyl-O—, wherein alkyl refers to an alkyl group, as defined above. Similarly, the terms “alkenyloxy,” “alkynyloxy,” and “cycloalkoxy,” refer to the groups alkenyl-O—, alkynyl-O—, and cycloalkyl-O—, respectively, wherein alkenyl, alkynyl, and cycloalkyl are as defined above. Examples of C₁-C₆-alkoxy groups include, but are not limited to, methoxy, ethoxy, C₂H₅—CH₂O—, (CH₃)₂CHO—, n-butoxy, C₂H₅—CH(CH₃)O—, (CH₃)₂CH—CH₂O—, (CH₃)₃CO—, n-pentoxy, 1-methylbutoxy, 2-methylbutoxy, 3-methylbutoxy, 1,1-dimethylpropoxy, 1,2-dimethylpropoxy, 2,2-dimethyl-propoxy, 1-ethylpropoxy, n-hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy, and 1-ethyl-2-methylpropoxy.

The term “alkylthio,” as used herein, refers to alkyl-S—, wherein alkyl refers to an alkyl group, as defined above. Similarly, the term “cycloalkylthio,” refers to cycloalkyl-S— where cycloalkyl are as defined above.

The term “alkylsulfinyl,” as used herein, refers to alkyl-S(O)—, wherein alkyl refers to an alkyl group, as defined above.

The term “alkylsulfonyl,” as used herein, refers to alkyl-S(O)₂—, wherein alkyl is as defined above.

The terms “alkylamino” and “dialkylamino,” as used herein, refer to alkyl-NH— and (alkyl)₂N— groups, where alkyl is as defined above.

The terms “alkylcarbonyl,” “alkoxycarbonyl,” “alkylaminocarbonyl,” and “dialkylaminocarbonyl,” as used herein, refer to alkyl-C(O)—, alkoxy-C(O)—, alkylamino-C(O)— and dialkylamino-C(O)— respectively, where alkyl, alkoxy, alkylamino, and dialkylamino are as defined above.

The term “heteroaryl,” as used herein, refers to a monovalent aromatic group of from 1 to 15 carbon atoms (e.g., from 1 to 10 carbon atoms, from 2 to 8 carbon atoms, from 3 to 6 carbon atoms, or from 4 to 6 carbon atoms) having one or more heteroatoms within the ring. The heteroaryl group can include from 1 to 4 heteroatoms, from 1 to 3 heteroatoms, or from 1 to 2 heteroatoms. In some examples, the heteroatom(s) incorporated into the ring are oxygen, nitrogen, sulfur, or combinations thereof. When present, the nitrogen and sulfur heteroatoms can optionally be oxidized. Heteroaryl groups can have a single ring (e.g., pyridyl or furyl) or multiple condensed rings provided that the point of attachment is through a heteroaryl ring atom. Preferred heteroaryls include pyridyl, piridazinyl, pyrimidinyl, pyrazinyl, triazinyl, pyrrolyl, indolyl, quinolinyl, isoquinolinyl, quinazolinyl, quinoxalinnyl, furanyl, thiophenyl, furyl, pyrrolyl, imidazolyl, oxazolyl, isoxazolyl, isothiazolyl, pyrazolyl benzofuranyl, and benzothiophenyl. Heteroaryl rings can be unsubstituted or substituted by one or more moieties as described for aryl above.

The term “alkylheteroaryl,” as used herein, refers to a heteroaryl group that is bonded to a parent compound through a diradical alkylene bridge, (—CH₂—)ₙ, where n is 1-12 and where “heteroaryl” is as defined above.

The terms “heterocyclyl,” “heterocyclic” and “heterocyclo” are used herein interchangeably, and refer to fully saturated or unsaturated, cyclic groups, for example, 3 to 7 membered monocyclic or 4 to 7 membered monocyclic; 7 to 11 membered bicyclic, or 10 to 15 membered tricyclic ring systems, having one or more heteroatoms within the ring.

The heterocyclyl group can include from 1 to 4 heteroatoms, from 1 to 3 heteroatoms, or from 1 to 2 heteroatoms. In some examples, the heteroatom(s) incorporated into the ring are oxygen, nitrogen, sulfur, or combinations thereof. When present, the nitrogen and sulfur heteroatoms can optionally be oxidized, and the nitrogen heteroatoms can optionally be quaternized. The heterocyclyl group can be attached at any heteroatom or carbon atom of the ring or ring system and can be unsubstituted or substituted by one or more moieties as described for aryl groups above.

Exemplary monocyclic heterocyclic groups include, but are not limited to, pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, 4-piperidonyl, pyridinyl, pyrazinyl, pyrimidinyl, pyridazinyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, triazolyl, triazinyl, and the like.

Exemplary bicyclic heterocyclic groups include, but are not limited to, indolyl, benzothiazolyl, benzoxazolyl, benzodioxolyl, benzothienyl, quinuclidinyl, quinolinyl, tetra-hydroisoquinolinyl, isoquinolinyl, benzimidazolyl, benzopyranyl, indolizinyl, benzofuryl, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]pyridinyl]or furo[2,3-b]pyridinyl), dihydroisoindolyl, dihydroquinazolinyl (such as 3,4-dihydro-4-oxo-quinazolinyl), tetrahydroquinolinyl and the like.

Exemplary tricyclic heterocyclic groups include carbazolyl, benzidolyl, phenanthrolinyl, acridinyl, phenanthridinyl, xanthenyl, and the like.

The term “alkylheterocyclyl,” as used herein, refers to a heterocyclyl group that is bonded to a parent compound through a diradical alkylene bridge, (—CH₂—)ₙ, where n is 1-12 and where “heterocyclyl” is as defined above. The term “heterocyclylalkyl,” as used herein, refers to a heterocyclyl group, as defined above, which is substituted by an alkyl group, as defined above.

Heretrocyclyl and heteroaryl groups can be unsubstituted or substituted with one or more moieties chosen from alkyl, halo, haloalkyl, hydroxyl, carboxyl, acyl, acyloxy, amino, alkyl- or dialkylamino, amido, arylamino, alkoxy, aryloxy, nitro, cyano, azido, thiol, imino, sulfonic acid, sulfate, sulfonyl, sulfanyl, sulfinyl, sulfamonyl, ester, phosphonyl, phosphinyl, phosphoryl, phosphine, thioester, thioether, acid halide, anhydride, oxime, hydrazine, carbamate, phosphoric acid, phosphate, phosphonate, or any other viable functional group that does not inhibit the biological activity of the compounds of the invention, either unprotected, or protected as necessary, as known to those skilled in the art, for example, as described in Greene, et al., Protective Groups in Organic Synthesis, John Wiley and Sons, Third Edition, 1999

The term “halogen,” as used herein, refers to the atoms fluorine, chlorine, bromine and iodine. The prefix halo- (e.g., as illustrated by the term haloalkyl) refers to all degrees of halogen substitution, from a single substitution to a perhalo substitution (e.g., as illustrated with methyl as chloromethyl (—CH₂Cl), dichloromethyl (—CHCl₂), trichloromethyl (—CCl₃)).

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

Reference will now be made in detail to specific aspects of the disclosed materials, compounds, compositions, articles, and methods, examples of which are illustrated in the accompanying Examples and Figures.

### Liposomal Formulations

The present disclosure provides liposomal formulations comprising (i) liposomes formed from a vesicle-forming lipid; (ii) a capturing agent-active agent complex encapsulated in the liposomes.

### Liposomes

The liposomes in the formulation can be composed primarily of vesicle-forming lipids. Such a vesicle-forming lipid is one that can form spontaneously into bilayer vesicles in water, as exemplified by the phospholipids, with its hydrophobic moiety in contact with the interior, hydrophobic region of the bilayer membrane, and its head group moiety oriented toward the exterior, polar surface of the membrane. Lipids capable of stable incorporation into lipid bilayers, such as cholesterol and its various analogs, can also be used in the liposomes. The vesicle-forming lipids are preferably lipids having two hydrocarbon chains, typically acyl chains, and a head group, either polar or nonpolar. There are a variety of synthetic vesicle-forming lipids and naturally-occurring vesicle-forming lipids, including the phospholipids, such as phosphatidylcholine, phosphatidylethanolamine, phosphatidylglycerol, phosphatidic acid, phosphatidylinositol, and sphingomyelin, where the two hydrocarbon chains are typically between about 14-22 carbon atoms in length, and have varying degrees of unsaturation. The above-described lipids and phospholipids whose acyl chains have varying degrees of saturation can be obtained commercially or prepared according to published methods. Other suitable lipids include glycolipids, cerebrosides and sterols, such as cholesterol.

The vesicle-forming lipid can be selected to achieve a specified degree of fluidity or rigidity, to control the stability of the liposome in serum, and/or to control the rate of release of the entrapped agent in the liposome. Liposomes having a more rigid lipid bilayer, or a gel-phase bilayer, are achieved by incorporation of a relatively rigid lipid, e.g., a lipid having a relatively high phase transition temperature, e.g., up to 60° C. Rigid, i.e., saturated, lipids contribute to greater membrane rigidity in the lipid bilayer. Other lipid components, such as cholesterol, are also known to contribute to membrane rigidity in lipid bilayer structures. On the other hand, lipid fluidity is achieved by incorporation of a relatively fluid lipid, typically one having a lipid phase with a relatively low gel to liquid-crystalline phase transition temperature, e.g., at or below room temperature.

The liposomes can optionally include a vesicle-forming lipid covalently linked to a hydrophilic polymer. As has been described, for example in U.S. Pat. No. 5,013,556, including such a polymer-derivatized lipid in the liposome composition forms a surface coating of hydrophilic polymer chains around the liposome. The surface coating of hydrophilic polymer chains is effective to increase the in vivo blood circulation lifetime of the liposomes when compared to liposomes lacking such a coating. Polymer-derivatized lipids comprised of methoxy(polyethylene glycol) (mPEG) and a phosphatidylethanolamine (e.g., dimyristoyl phosphatidylethanolamine, dipalmitoyl phosphatidylethanolamine, distearoyl phosphatidylethanolamine (DSPE), or dioleoyl phosphatidylethanolamine) can be obtained from Avanti Polar Lipids, Inc. (Alabaster, Ala.) at various mPEG molecular weights (350, 550, 750, 1,000, 2,000, 3,000, and 5,000 Daltons). Lipopolymers of mPEG-ceramide can also be purchased from Avanti Polar Lipids, Inc. Preparation of lipid-polymer conjugates is also described in the literature, see U.S. Pat. Nos. 5,631,018, 6,586,001, and 5,013,556; Zalipsky, S. et al., Bioconjugate Chem. 8:111 (1997); Zalipsky, S. et al., Meth. Enzymol. 387:50 (2004). These lipopolymers can be prepared as well-defined, homogeneous materials of high purity, with minimal molecular weight dispersity (Zalipsky, S. et al., Bioconjugate Chem. 8:111 (1997); Wong, J. et al., Science 275:820 (1997)). The lipopolymer can also be a “neutral” lipopolymer, such as a polymer-distearoyl conjugate, as described in U.S. Pat. No. 6,586,001, incorporated by reference herein.

When a lipid-polymer conjugate is included in the liposomes, typically between 1-20 mole percent of the lipid-polymer conjugate is incorporated into the total lipid mixture (see, e.g., U.S. Pat. No. 5,013,556).

In some embodiments, the vesicle-forming lipids may include a lipid, a phospholipid, and a PEG-conjugated phospholipid. The lipid may be selected from the group consisting of fatty acids, lysolipids, sphingolipids, glycolipids, glycosphingolipids, phospholipids, glycerolipids, glycerophospholipids, triacylglycerols, sterol lipids such as cholesterol, and prenol lipids. In some embodiments, the liposome may include a lipid such as cholesterol. In some embodiments, the liposome may comprise a phospholipid selected from a phosphatidylcholine (PC), a phosphatidylethanolamine (PE), a phosphatidylserine (PS), a phosphatidylinositol (PI), a phosphatidic acid (PA), a phosphatidylglycerol (PG), and a cardiolipid (CL). In some embodiments, the glycerophospholipid may be a dimyristoyl, a dipalmitoyl, or a distearoyl glycerophospholipid.

In some embodiments, the phospholipid may be selected from 1,2-distearoyl-sn-glycero-3-phosphocholine (DSPC), 1,2-distearoyl-sn-glycero-3-phosphoethanolamine (DSPE), 1,2-dipalmitoyl-sn-glycero-3-phosphocholine (DPPC), hydrogenated soybean phosphatidylcholine (HSPC), or sphingomyelin (SPH), dicetylphophosphate,1,2-dimyristoyl-sn-glycero-3-phosphocholine (DMPC), 1,2-dioleoyl-sn-glycero-3-phosphocholine (DOPC), 1,2-dioleoyl-sn-glycero-3-phosphoethanolamine (DOPE), 1-palmitoyl-2-oleoyl-sn-glycero-3-phosphocholine (POPC), 1,2-distearoyl-sn-glycero-3-phosphoglycerol (DSPG), 1,2-dipalmitoyl-sn-glycero-3-phosphoglycerol (DPPG), 1,2-dioleoyl-sn-glycero-3-phospho-L-serine (DOPS), 1,2-dioleoyl-sn-glycero-3-phosphoglycerol (DOPG), 1,2-dipalmitoyl-sn-glycero-3-phosphoethanolamine (DPPE), 1-palmitoyl-2-oleoyl-sn-glycero-3-phosphoglycerol (POPG), 1,2-dieurocoyl-sn-glycero-3-phosphocholine (DEPC), 1-myristoryl-2-stearoyl-sn-glycero-3-phosphocholine (MSPC), 1,2-dierucoyl-sn-glycero-3-phosphoethanolamine (DEPE), 1-palmitoyl-2-oleoyl-sn-glycero-3-phosphoethanolamine (POPE), 1-palmitoyl-2-oleoyl-sn-3-phospho-L-serine (POPS), 1,2-distearoyl-sn-glycero-3-phospho-L-serine (DSPS), 1,2-dimyristoyl-sn-glycero-3-phosphoglycerol (DMPG), 1,2-dipalmitoyl-sn-glycero-3-phosphatidic acid (DPPA), 1,2-dimyristoyl-sn-glycero-3-phosphoethanolamine (DMPE), 1,2-dimyristoyl-sn-glycero-3-phospho-L-serine (DMPS), 1-stearoyl-2-lyso-sn-glycero-phosphocholine (S-LysoPC), 1,2-dipalmitoyl-sn-glycero-3-phospho-L-serine (DPPS), 1-stearoyl-2-oleoyl-sn-glycero-3-phosphocholine (SOPC), 1,2-dilinoleoyl-sn-glycero-3-phosphocholine (DLoPC), 1,2-didecanoyl-sn-glycero-3-phosphocholine (DDPC), 1-palmitoyl-2-lyso-sn-glycero-phosphocholine (P-LysoPC), 1-palmitoyl-2-stearoyl-sn-glycero-3-phosphocholine (PSPC), 1,2-dilauroyl-sn-glycero-3-phosphoethanolamine (DLPE), 1-myristoyl-2-palmitoyl-sn-glycero-3-phosphocholine (MPPC), 1-palmitoyl-2-myristoyl-sn-glycero-3-phosphocholine (PMPC), 1,2-distearoyl-sn-glycero-3-phosphatidic acid (D SPA), 1,2-dilinoleol-sn-glycero-3-phosphoethanolamine (DLoPE), 1-myristoyl-2-lyso-sn-glycero-3-phosphocholine (M-LysoPC), 1,2-dimyristoyl-sn-glycero-3-phosphatidic acid (DMPA), 1-oleoyl-2-lyso-sn-glycerophosphocholine (O-LysoPC), salts thereof (such as, for example, sodium salts or ammonium salts), and combinations thereof.

In some embodiments, the phospholipid can be conjugated to PEG itself or a copolymer containing PEG, such as PEO-PPO copolymer available under the tradename PLURONIC. The PEG conjugated phospholipid can be selected from 1,2-dimyristoyl-rac-glycero-3-methylpolyoxyethylene (DMG-PEG 2000 or DMG-PEG 5000), N-(methylpolyoxyethylene oxycarbonyl)-1,2-distearoyl-sn-glycero-3-phosphoethanolamine (DSPE-PEG or DSPE-PEG), 1,2-distearoyl-rac-glycero-3-methylpolyoxyethylene (DSG-PEG or DSG-PEG), N-(methylpolyoxyethylene oxycarbonyl)-1,2-dimyristoyl-sn-glycero-3-phosphoethanolamine (DMPE-PEG), N-(methylpolyoxyethylene oxycarbonyl)1,2-dipalmitoyl-sn-glycero-3-phosphoethanolamine (DPPE-PEG 2000 or DPPE-PEG 5000), N-[2′,3′-bis(metylpolyoxyethylene oxy)propane-1′-oxycarbonyl]-1,2-distearoyl-sn-glycero-3-phosphoethanolamine (DSPE-2arm-PEG or DSPE-2arm-PEG), 1,2-dipalmitoyl-rac-glycero-3-methylpolyoxyethylene (DPG-PEG or DPG-PEG), 1,2-dioleoyl-rac-glycerol methoxypolyethylene glycol (DOG-PEG or DOG-PEG), salts thereof (such as, for example, sodium salts or ammonium salts), or combinations thereof. The molecular weight of PEG can vary. In some embodiments, the molecular weight of PEG is from about 500 Daltons to about 10,000 Daltons, preferably from about 500 Daltons to about 5,000 Daltons, more preferably from about 1,000 Daltons to about 5,000 Daltons, most preferably from about 2,000 Daltons to about 4,000 Daltons. In some embodiments, the molecular weight of PEG is about 2000 Daltons.

In some embodiments, the liposomes are PEG-free and/or cholesterol-free. In some embodiments, the liposomes can be anionic or cationic liposomes comprising charged lipids in a wide range of compositions. For example, in some embodiments, anionic liposomes can be used to selectively target myeloid cells and bone marrow (e.g., in AML therapy) or macrophages.

In some embodiments, when the capturing agent-active agent complex has an anionic moiety the lipid can be a cationic lipid (e.g., stearylamine or DOTAP). In some embodiments, drug incorporation can be stabilized by the presence of a cationic lipid due to its ability to ion pair with the anionic moiety of the capturing agent-active agent complex.

In some embodiments, the phospholipid, the lipid, and the PEG-phospholipid are present in a molar percent ratio ranging from about 50:25:25 to about 90:5:5, such as in a molar percent ratio of about 60:35:5. On the other hand, the inclusion of a PEG-phospholipid or cholesterol is optional.

In some embodiments, the phospholipid, the lipid, capturing agent, and the PEG-phospholipid are present in a molar percent ratio ranging from about 50:25:20:5 to about 85:5:5:5, such as in a molar percent ratio of about 50:25:20:5 or about 60:15:20:5.

In some embodiments, the liposome has a mean particle size (diameter) ranging from 45 nm to 30 μm. For example from 50 nm to 140 nm, from 50 nm to 250 nm, or from 60 to 140 nm, such as about 50 nm, about 60 nm, about 70 nm, about 80 nm, about 90 nm, about 100 nm, about 110 nm, about 120 nm, about 130 nm, about 140 nm, about 150 nm, about 180, about 200 nm, about 220 nm, or about 250 nm, about 500 nm, about 1 μm, about 5 μm, about 10 μm, about 20 μm, or about 30 μm.

If desired for a particular application, the liposomes can include a ligand, such as a targeting ligand, conjugated to the liposome. For example, the liposomes can optionally include a lipopolymer modified to include a ligand, forming a lipid-polymer-ligand conjugate, also referred to herein as a ‘lipopolymer-ligand conjugate’. The ligand can be a therapeutic molecule, such as a drug or a biological molecule having activity in vivo, a diagnostic molecule, such as a contrast agent or a biological molecule, or a targeting molecule having binding affinity for a binding partner, preferably a binding partner on the surface of a cell. For example, the ligand can have binding affinity for the surface of a cell, so as to facilitate entry of the liposome into the cytoplasm of a cell via internalization. A ligand present in liposomes that include such a lipopolymer-ligand is oriented outwardly from the liposome surface, and therefore available for interaction with its cognate receptor.

Methods for attaching ligands to lipopolymers are known, where the polymer can be functionalized for subsequent reaction with a selected ligand. (U.S. Pat. No. 6,180,134; Zalipsky, S. et al., FEBS Lett. 353:71 (1994); Zalipsky, S. et al., Bioconjugate Chem. 4:296 (1993); Zalipsky, S. et al., J. Control. Rel. 39:153 (1996); Zalipsky, S. et al., Bioconjugate Chem. 8(2):111 (1997); Zalipsky, S. et al., Meth. Enzymol. 387:50 (2004)). Functionalized polymer-lipid conjugates can also be obtained commercially, such as end-functionalized PEG-lipid conjugates (Avanti Polar Lipids, Inc.). The linkage between the ligand and the polymer can be a stable covalent linkage or a releasable linkage that is cleaved in response to a stimulus, such as a change in pH or presence of a reducing agent.

The ligand can be a molecule that has binding affinity for a cell receptor or for a pathogen circulating in the blood. The ligand can also be a therapeutic or diagnostic molecule, in particular molecules that when administered in free form have a short blood circulation lifetime. In one embodiment, the ligand is a biological ligand, such as a ligand having binding affinity for a cell receptor. Example biological ligands include molecules having binding affinity to receptors for CD4, folate, insulin, LDL, vitamins, transferrin, asialoglycoprotein, selectins, such as E, L, and P selectins, Flk-1,2, FGF, EGF, integrins, in particular, α₄β₁ αᵥβ₃, αᵥβ₁ αᵥβ₅, αᵥβ₆ integrins, HER2, and others. Ligands are known in the art, and can include proteins and peptides, including antibodies and antibody fragments, such as F(ab′)₂, F(ab)₂, Fab′, Fab, Fv (fragments consisting of the variable regions of the heavy and light chains), and scFv (recombinant single chain polypeptide molecules in which light and heavy variable regions are connected by a peptide linker), and the like. The ligand can also be a small molecule peptidomimetic. It will be appreciated that a cell surface receptor, or fragment thereof, can serve as the ligand. Other example targeting ligands include, but are not limited to vitamin molecules (e.g., biotin, folate, cyanocobalamine), oligopeptides, oligosaccharides. Other example ligands include those described in U.S. Pat. Nos. 6,214,388, 6,316,024, 6,056,973, and 6,043,094, which are herein incorporated by reference.

Liposomal formulations including the complex described herein can be formed using any suitable method for preparing and/or loading liposomes. For example, a complex described herein and one or more vesicle-forming lipids can be dissolved in a suitable solvent, and the solvent can be evaporated to form a lipid film. The lipid film can be hydrated with an aqueous solution (e.g., having a pH of from 7-9) to form liposomes comprising the entrapped complex.

After liposome formation, the liposomes can be sized to obtain a population of liposomes having a substantially homogeneous size range, for example from 0.01 to 0.5 microns (e.g., from 0.03-0.40 microns). Liposomes can be sized by any suitable method, such as by extrusion through a series of membranes having a selected uniform pore size (e.g., polycarbonate membranes having a selected uniform pore size in the range of 0.03 to 0.2 micron). The pore size of the membrane corresponds roughly to the largest sizes of liposomes produced by extrusion through that membrane, particularly where the preparation is extruded two or more times through the same membrane. Homogenization methods can also be used to prepare liposomes having sizes of 100 nm or less (Martin, F. J., in Specialized Drug Delivery Systems—Manufacturing and Production Technology, P. Tyle, Ed., Marcel Dekker, New York, pp. 267-316 (1990)).

In some embodiments, the liposomes in the formulation can have an average particle size, as measured by dynamic light scattering, of from 45 nm to 30 μm (e.g., from 45 nm to 1 μm, from 50 nm to 500 nm, from 50 nm to 250 nm, from 50 nm to 200 nm, from 75 nm to 150 nm, from 80 nm to 110 nm, from 90 nm to 150 nm, from 120 nm to 150 nm, from 100 nm to 130 nm, or from 90 nm to 110 nm). In some embodiments, the liposomes in the formulation can have a zeta potential of from −50 mV to 0 mV (e.g., from −25 mV to 0 mV, from −15 mV to 0 mV, or from −10 mV to 0 mV).

After sizing, unencapsulated compound can be removed by a suitable technique, such as dialysis, centrifugation, tangential-flow diafiltration, size exclusion chromatography or ion exchange to achieve a suspension of liposomes having a high concentration of entrapped compound in the liposomes and little to no compound in solution outside of the liposomes. Also after liposome formation, the external phase of the liposomes can be adjusted, if desired, by titration, dialysis or the like, to an appropriate pH.

Once formed, the liposomal suspension can be lyophilized using methods known in the art. The resulting composition can be in the form of a lyophilized powder. The term “lyophilized powder” refers to any solid material obtained by lyophilization of an aqueous mixture. In some examples, a lyoprotectant, such as sucrose or trehalose, can be added to the liposomal formulation prior lyophilization.

Stability of the lyophilized formulation can be assessed by visual inspection for appearance of cake, the time of reconstitution, and the property of the reconstituted liposomes after various lengths of storage time. In terms of quantitative standard, a liposomal formulation can be assessed for appearance of particulates or turbidity by visual inspection, change in color, mean particle size and polydispersity index by dynamic light scattering on a particle size analyzer (e.g., using a NICOMP370 particle sizing system), zeta potential measurement (e.g., using Malvern Instrument's Zetasizer), percentage of drug encapsulation by chromatography (e.g., by size-exclusion chromatography on a Sepharose CL-4B column), chemical integrity of the drug substance and of excipients and appearance of decomposition products by HPLC and by LC-MS. The preferred stability range for the BTZ liposome formulation is less than 20% change in mean particle size and drug encapsulation percentage after 6 months storage at 4 degree compared to immediately reconstituted sample; less than 5% chemical decomposition of the drug product.

Lyophilized formulations can be readily reconstituted prior to administration by adding an aqueous solvent. The reconstitution solvent can be suitable for pharmaceutical administration (e.g., for parenteral administration to a subject). Examples of suitable reconstitution solvents include, without limitation, water, saline, and phosphate buffered saline (PBS).

The liposomal formulation described herein can be used in combination with other anticancer agents, including various chemotherapeutic agents, such as cytarabine, fludarabine, decitabine, daunorubicin, and therapeutic antibodies such as rituximab, alemtuzumab, tyrosine kinase inhibitors, and any other agents with anticancer activities. In addition, anticancer agents can be co-formulated in the same liposome formulation as the boronic acid agent. For example, daunorubicin or cytarabine can be co-encapsulated into liposomal bortezomib at a defined drug ratio. Such type of combination can mediate co-delivery of the therapeutic combination at a ratio that produces optimal therapeutic synergism.

In some embodiments, the liposomal formulations described herein release the active agent loaded therein in a pH-dependent manner. Upon exposure to an environment having an acidic pH, active agent-capturing agent complex dissociates, allowing the release of the active agent loaded therein.

In some embodiments, the liposomes release at least about 10% of the loaded active agent within 60 minutes at a pH of 5. In some embodiments, the liposomes release at least about 20% or more of the loaded active agent within 30 minutes at a pH of 5

In some embodiments, the rate of active agent release can be tuned by the concentration of the complexing agent (e.g., tiron), as well as a buffering agent (e.g., phosphate buffer or glycine buffer at pH 8). To create an inside high pH gradient, calcium acetate or sodium carbonate can be used for the internal phase during liposome preparation. The inside pH must be >6, preferably >7 to ensure good stability of the BTZ complex. Higher pH and greater buffering will reduce drug release in vivo, although the pH is limited by the hydrolysis of lipids at very high pH values. The release rate is tunable by adjusting lipid composition and the concentration of capturing agent and buffering agent inside the liposome as well as the pH. The active agent release in vivo is affected by plasma protein binding, lipid degradation, extraction, macrophage uptake, and plasma environment. For example, unionized carbonic acid can diffuse into the liposomes and lower internal pH and disrupt BTZ complex. Depending on the biological readout, the release rate can be tuned to achieve the best balance between efficacy and toxicity.

### Capturing Agent-Active Agent Complex

In some embodiments the liposomal formulation can include boronic acid esters of boronic acid therapeutic agents. The boronic acid esters can function as prodrugs for the boronic acid therapeutic agents. The boronic acid esters can be used to prepare liposomal formulations with improved properties, such as enhanced stability.

In some embodiments, the capturing agent-active agent complex is a compound defined by Formula A:

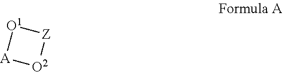

or a pharmaceutically acceptable salt thereof,
wherein:
A represents an active agent comprising a boronic acid, and
Z together with O¹ and O² represent a capture agent, wherein Z comprises an aromatic diol substituted one or more charged moieties,
wherein A is bound to the capture agent so as to form a boronic ester having the structure below

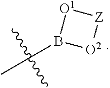

In some embodiments, the capturing agent-active agent complex is a compound defined by Formula A-1:

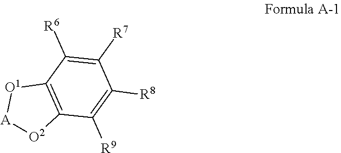

or a pharmaceutically acceptable salt thereof,
wherein:
A represents an active agent comprising a boronic acid, and
R⁶-R⁹ are independently hydrogen, oxo, hydroxy, carboxylic acid, carboxylate, sulfonic acid, sulfonate, phosphoryl group, a primary amine, a secondary amine, a tertiary amine, quaternary amine, substituted or unsubstituted alkyl group, cycloalkyl group, heterocyclyl group, aryl group, heteroaryl group, alkylaryl group, arylalkyl group, alkylcycloalkyl group, alkylheterocyclyl group, alkylheteroaryl group, alkoxy group, alkylthio group, or alkylamino group; and at least one of R⁶-R⁹ is charged moiety;
wherein A is bound to O¹ and O² so as to form a boronic ester having the structure below

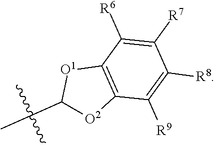

In some embodiments, the capturing agent-active agent complex is a compound defined by Formula A-2:

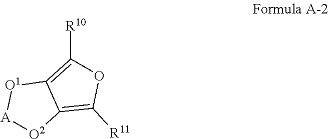

or a pharmaceutically acceptable salt thereof,
wherein:
A represents an active agent comprising a boronic acid, and
R¹⁰ and R¹¹ are independently hydrogen, oxo, hydroxy, carboxylic acid, carboxylate, sulfonic acid, sulfonate, phosphoryl group, a primary amine, a secondary amine, a tertiary amine, quaternary amine, substituted or unsubstituted alkyl group, cycloalkyl group, heterocyclyl group, aryl group, heteroaryl group, alkylaryl group, arylalkyl group, alkylcycloalkyl group, alkylheterocyclyl group, alkylheteroaryl group, alkoxy group, alkylthio group, or alkylamino group; and at least one of R¹⁰ or R¹¹ is an charged moiety;
wherein A is bound to O¹ and O² so as to form a boronic ester having the structure below

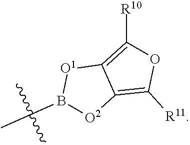

In some embodiments, the capturing agent-active agent complex is a compound defined by Formula A-3:

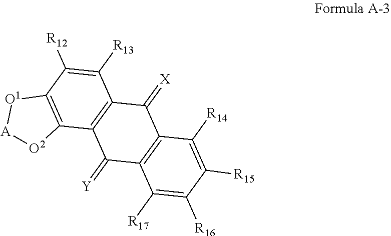

or a pharmaceutically acceptable salt thereof,
wherein:
A represents an active agent comprising a boronic acid, and
R¹²-R¹⁷ are independently hydrogen, oxo, hydroxy, carboxylic acid, carboxylate, sulfonic acid, sulfonate, phosphoryl group, a primary amine, a secondary amine, a tertiary amine, quaternary amine, substituted or unsubstituted alkyl group, cycloalkyl group, heterocyclyl group, aryl group, heteroaryl group, alkylaryl group, arylalkyl group, alkylcycloalkyl group, alkylheterocyclyl group, alkylheteroaryl group, alkoxy group, alkylthio group, or alkylamino group; and at least one of R¹²-R¹⁷ is a charged moiety;
wherein A is bound to O¹ and O² so as to form a boronic ester having the structure below

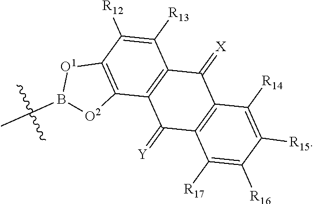

Suitable boronic acid containing active agent can include, but are not limited to: a boronic acid containing anticancer agent, a boronic acid containing antimicrobial agent, a boronic acid containing antiproliferative agent, a boronic acid containing antibiotic agent, a boronic acid containing antimitotic agent, a boronic acid containing antiviral agent, a containing boronic acid prodrug (e.g., boronic acid containing camptothecin prodrug, boronic acid containing methotrexate prodrug, or boronic acid containing crizotinib prodrug), a boronic acid containing proteasome inhibitor, a boronic acid containing autotaxin inhibitor, an urea-containing peptide boronic acid, a boronic acid containing histone deacetylases inhibitor, a boronic acid β-lactamase inhibitor, a boronic acid containing sensor (e.g., boronic acid containing photo induced electron transfer materials), a poly(aniline boronic acid) polymers, a boronic acid containing carbohydrate sensor, a boronic acid containing dopamine sensor, a boronic acid containing cholesterol analog, a boronic acid containing chalcone, a boronic acid containing peptide, or any combination thereof.

In certain examples, the capturing agent-active agent complex of Formula A is a compound as follows:

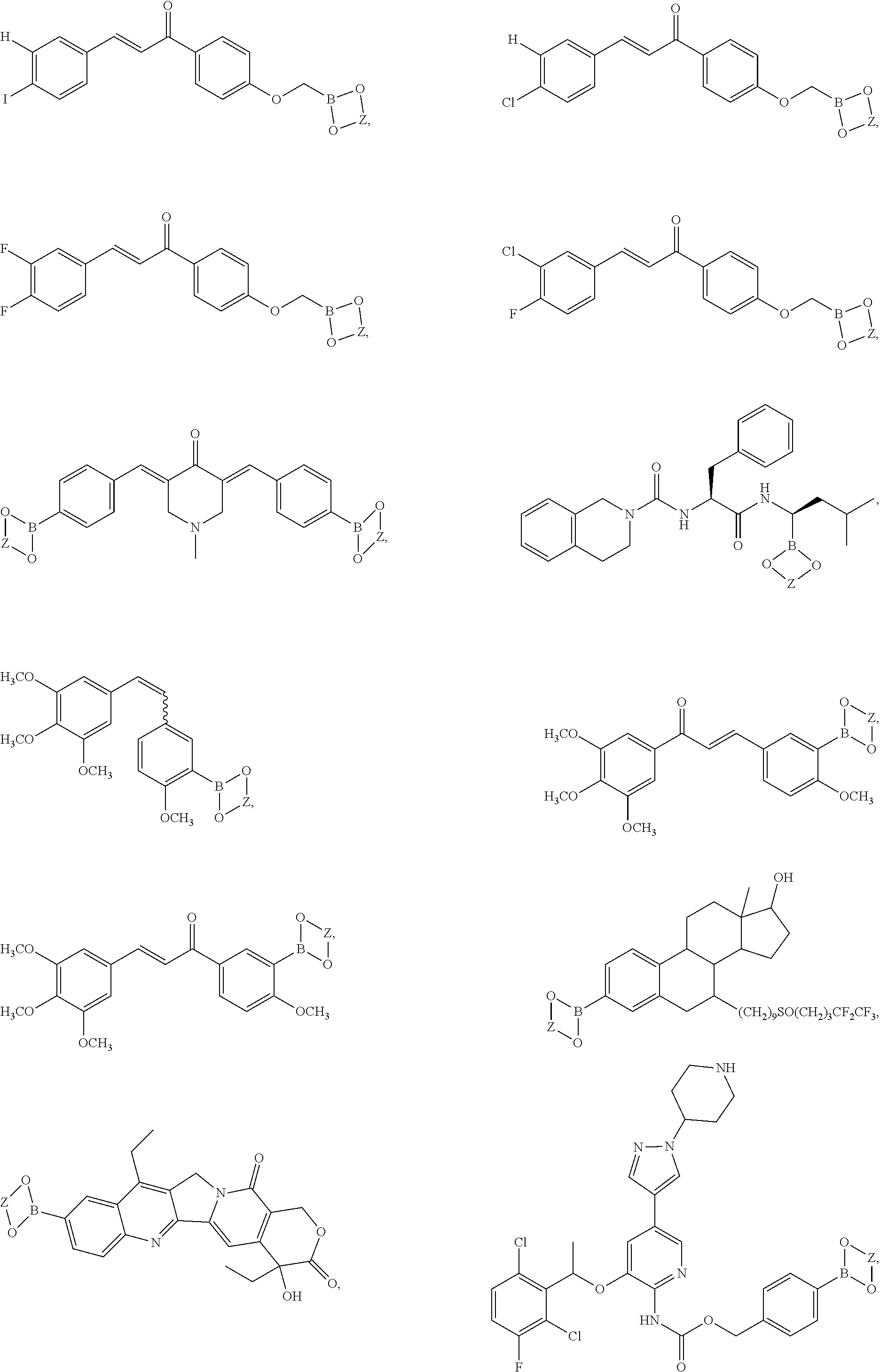

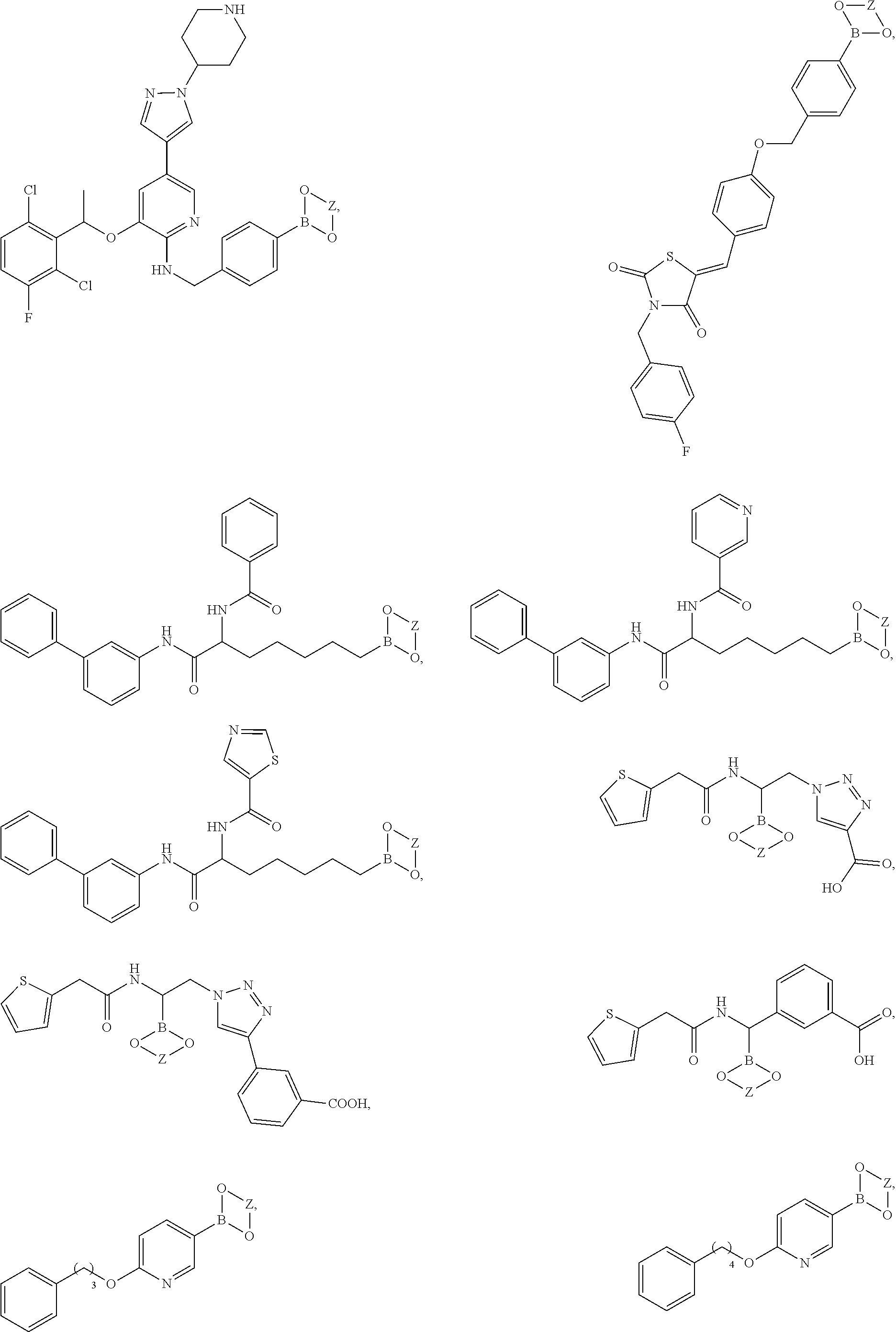

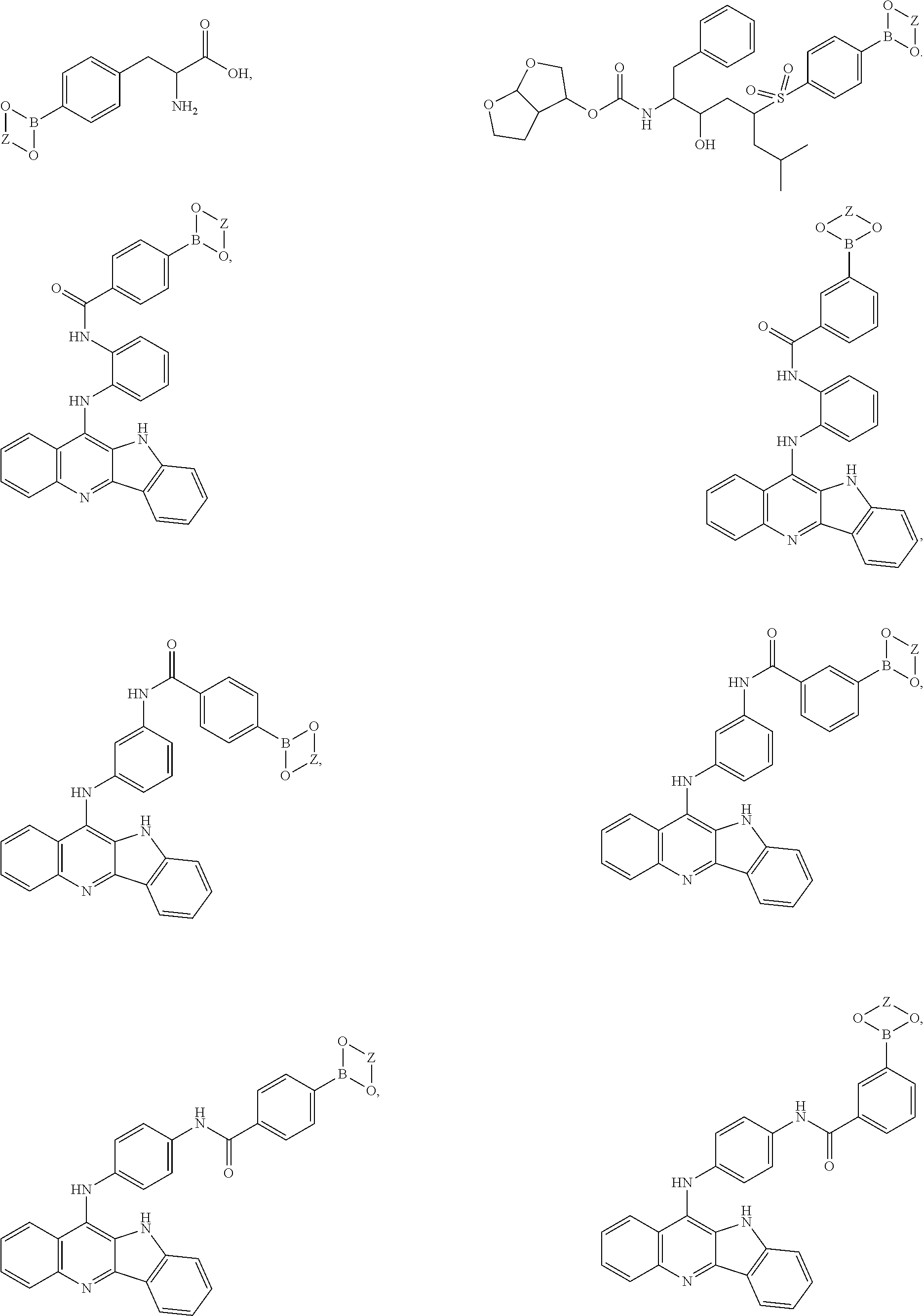

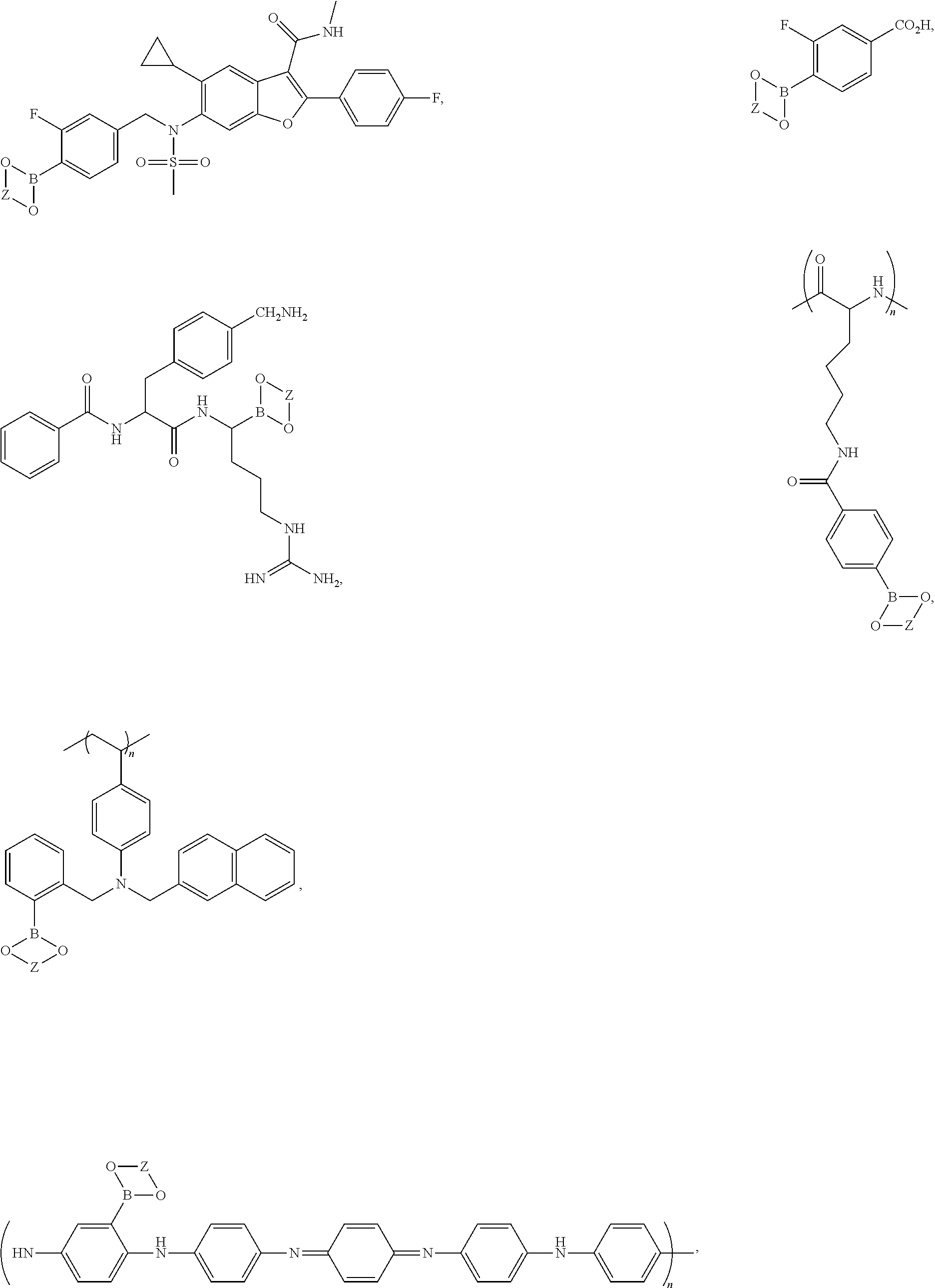

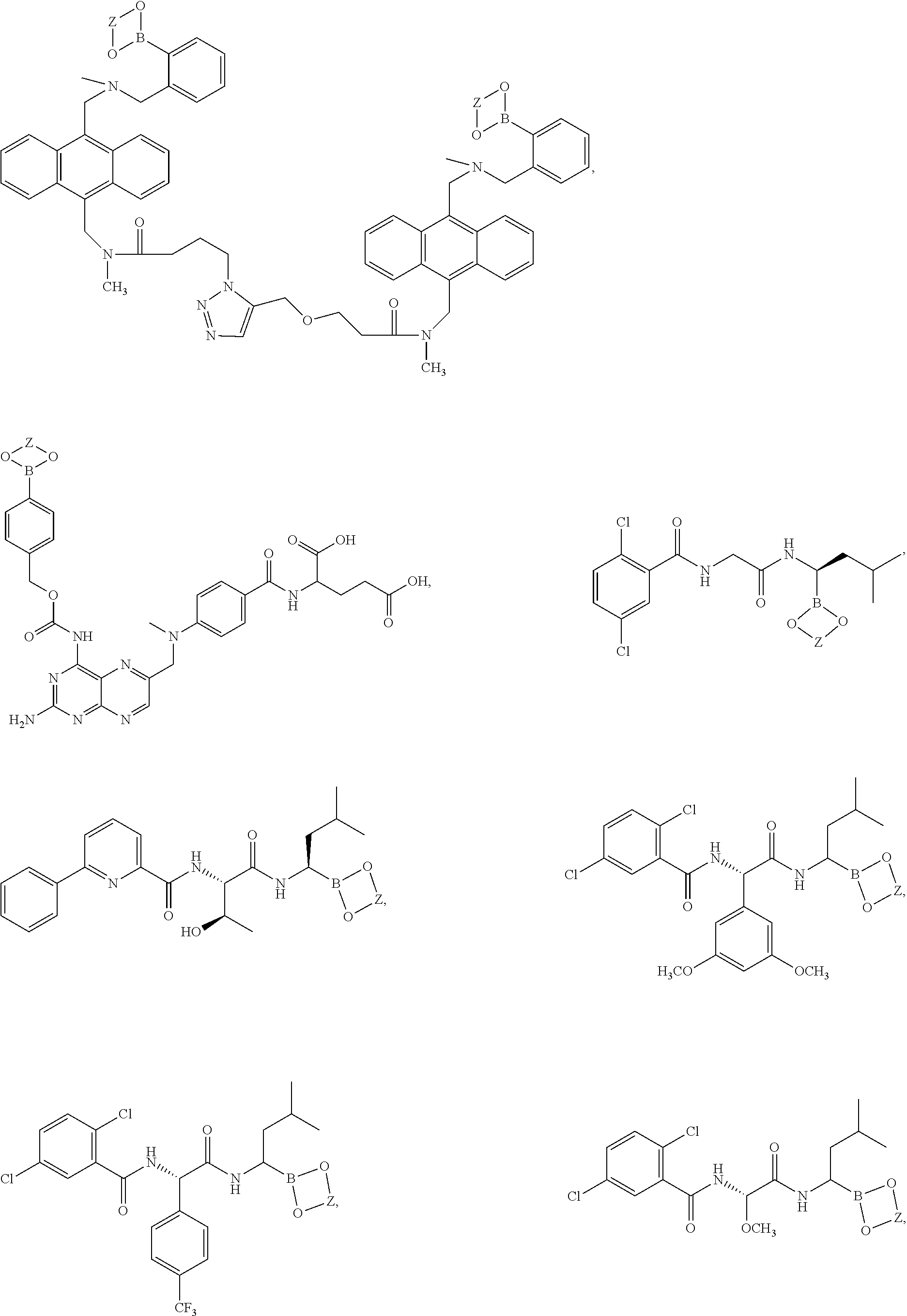

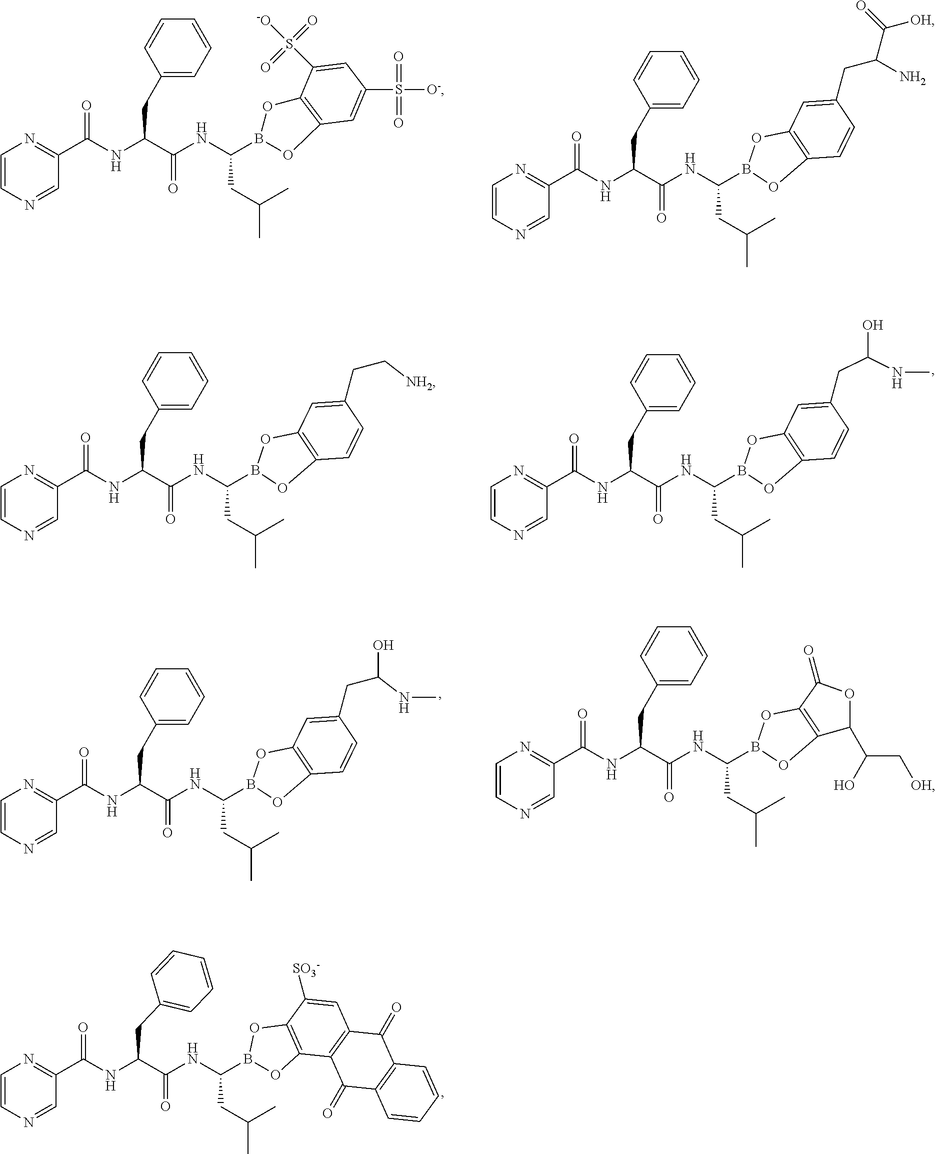

or a pharmaceutically acceptable salt thereof.

In some embodiments, the capturing agent-active agent complex is a compound defined by Formula I:

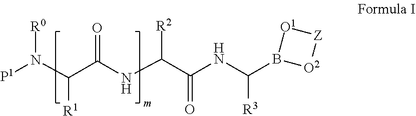

wherein
P¹ is hydrogen or an amino-group protecting moiety;
R⁰ is hydrogen or an alkyl group;
R¹, R², and R³ are independently hydrogen, an alkyl group, a cycloalkyl group, a heterocyclyl group, an aryl group, a heteroaryl group, or —CH₂—R⁴;
R⁴ is an aryl group, an alkylaryl group, an arylalkyl group, a cycloalkyl group, an alkylcycloalkyl group, a heterocyclyl group, an alkylheterocyclyl group, a heteroaryl group, an alkylheteroaryl group, an alkoxy group, or an alkylthio group;
m is 0, 1, or 2;
Z, together with O¹ and O², represent a capture agent, wherein Z comprises one or more charged moieties; or
a pharmaceutically acceptable salt thereof.

In some embodiments, the one or more charged moieties can comprise an anionic moiety (e.g., from one to three anionic moieties). In some embodiments, the one or more charged moieties can comprise a cationic moiety (e.g., from one to three cationic moieties). In some embodiments, the one or more charged moieties can comprise one or more anionic moieties and one or more cationic moieties.

In some examples, P¹ is an amino-group protecting moiety. Amino-group protecting moieties include groups that are used to derivatize an amino group, especially an N-terminal amino group of a peptide or amino acid. Such groups include, without limitation, alkyl, acyl, alkoxycarbonyl, aminocarbonyl, and sulfonyl moieties. However, the term “amino-group protecting moiety” is not intended to be limited to those particular protecting groups that are commonly employed in organic synthesis, nor is it intended to be limited to groups that are readily cleavable.

In some examples, P¹ is R⁵—C(O)—, R⁵—S(O)₂—, R⁵—NH—C(O)—, or R⁵—O—C(O)—, wherein R⁵ is an alkyl group, an aryl group, an alkylaryl group, an arylalkyl group, a cycloalkyl group, an alkylcycloalkyl group, a heterocyclyl group, an alkylheterocyclyl group, a heteroaryl group, or an alkylheteroaryl group. In certain examples, P¹ is R⁵—C(O)—, R⁵—S(O)₂—, R⁵—NH—C(O)—, or R⁵—O—C(O)—, and R⁵ is a heteroaryl group. In certain examples, P¹ is (2-pyrazine)carbonyl.

In some examples, R⁰ is hydrogen. In other examples, R⁰ is an alkyl group. For example, R⁰ can be a C₁-C₆ alkyl group. In some examples, R⁰ can be a C₁-C₄ alkyl group. In certain examples, R⁰ can be methyl or ethyl.

In some examples, R¹, R², and R³ are each independently chosen from hydrogen, C₁-C₈ alkyl, C₃-C₁₀ cycloalkyl, C₆-C₁₀ aryl, and —CH₂—R⁴, wherein each of R¹, R², R³, and R⁴ can be optionally substituted as described above. In some examples, R¹, R², and R³ are each independently chosen from C₁-C₄ alkyl and —CH₂—R⁴, and R⁴ is one of cycloalkyl, aryl, heterocyclyl, heteroaryl, or alkoxy. In some examples, R¹, R², and R³ are each independently chosen from C₁-C₄ alkyl and —CH₂—R⁴, and R⁴ is one of C₆-C₁₀ aryl, (C₆-C₁₀)ar(C₁-C₆)alkyl, (C₁-C₆)alk(C₆-C₁₀)aryl, C₃-C₁₀ cycloalkyl, C₁-C₈ alkoxy, or C₁-C₈, alkylthio or a 5- to 10-membered heteroaryl ring. In certain examples, R³ is an isobutyl group.

m can be 0, 1, or 2. When m is zero, the residue within the brackets is not present, and the boronate ester compound is a dipeptide. Similarly, when m is 1, the residue within the brackets is present, and the compound is a tripeptide. When m is 2, the compound is a tetrapeptide. In certain examples, m is zero. The terms “peptide,” ‘dipeptide,” and “tripeptide,” as used here, are intended to encompass compounds comprising natural amino acid residues, unnatural amino acid residues, or a combination of natural and unnatural amino acid residues. It will be apparent that the terms “peptide,” “dipeptide,” and “tripeptide” are used to refer to compounds herein in which the carboxylic acid functionality of the C-terminal amino acid residue is replaced by a boronate ester functionality.

The charged moieties can be anionic, cationic, or zwitterionic moieties. The charged moieties can be attached to the boron atom by two hydroxyl groups of the capture agent, such that the resulting boronate ester forms a 5-membered ring. In certain examples, the capture agent can comprise a 1,2-diol. For example, the capture agent can comprise an aromatic diol (e.g., a catechol or catechol derivative, or a furan diol substituted with one or more charged moieties).

When present, the anionic moiety can comprise any suitable negatively charged moiety (e.g., a sulfonate, carboxylate, phosphoryl group). When present, the cationic moiety can comprise any suitable positively charged moiety (e.g., an amino group, a secondary amine, a tertiary amine, or a quaternary amine).

In some examples, the capture agent can comprise a diol (e.g., substituted with one or more charged moieties). In some examples, the capture agent can comprise an aromatic diol (e.g., a catechol or catechol derivative, or a furan diol). In some embodiments, the capture agent can comprise a catechol, such as tiron or a derivative thereof. In some embodiments, the capture agent can comprise a furan diol, such as ascorbic acid or a derivative thereof. In some examples, the capture agent can be a catechol such as a catecholamine (e.g., dopamine, epinephrine, norepinephrine, or a derivative thereof). In some embodiments, the capture agent can comprise a cationic moiety which can stabilize BTZ complex by ion pairing with the boronic ester (anionic), thereby further stabilizing the complex.

In certain examples, wherein the capturing agent-active agent complex is a compound defined by Formula IA

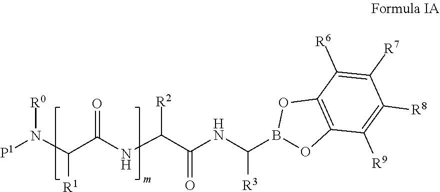

wherein P¹, R⁰, R¹, R², R³, R⁴, and m, are as defined above with respect to Formula I, and R⁶-R⁹ are independently hydrogen, oxo, hydroxy, carboxylic acid, carboxylate, sulfonic acid, sulfonate, phosphoryl group, a primary amine, a secondary amine, a tertiary amine, quaternary amine, substituted or unsubstituted alkyl group, cycloalkyl group, heterocyclyl group, aryl group, heteroaryl group, alkylaryl group, arylalkyl group, alkylcycloalkyl group, alkylheterocyclyl group, alkylheteroaryl group, alkoxy group, alkylthio group, or alkylamino group; and at least one of R⁶-R⁹ is an charged moiety; or
a pharmaceutically acceptable salt thereof.

In some embodiments, each charged moiety can comprise an anionic moiety. In some embodiments, each charged moiety can comprise a cationic moiety. In some embodiments, at least one of R⁶-R⁹ can comprise an anionic moiety and at least one of R⁶-R⁹ can comprise a cationic moiety

In certain examples, the capturing agent-active agent complex is a compound defined by Formula IB

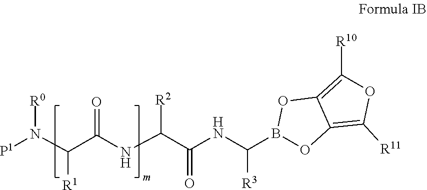

wherein
P¹, R⁰, R¹, R², R³, R⁴, and m are as defined above with respect to Formula I,
R¹⁰ and R¹¹ are independently hydrogen, oxo, hydroxy, carboxylic acid, carboxylate, sulfonic acid, sulfonate, phosphoryl group, a primary amine, a secondary amine, a tertiary amine, quaternary amine, substituted or unsubstituted alkyl group, cycloalkyl group, heterocyclyl group, aryl group, heteroaryl group, alkylaryl group, arylalkyl group, alkylcycloalkyl group, alkylheterocyclyl group, alkylheteroaryl group, alkoxy group, alkylthio group, or alkylamino group; and at least one of R¹⁰ or R¹¹ is a charged moiety; or a pharmaceutically acceptable salt.

In some embodiments, each charged moiety can comprise an anionic moiety. In some embodiments, each charged moiety can comprise a cationic moiety

In certain example, the capturing agent-active agent complex is a compound as follows:

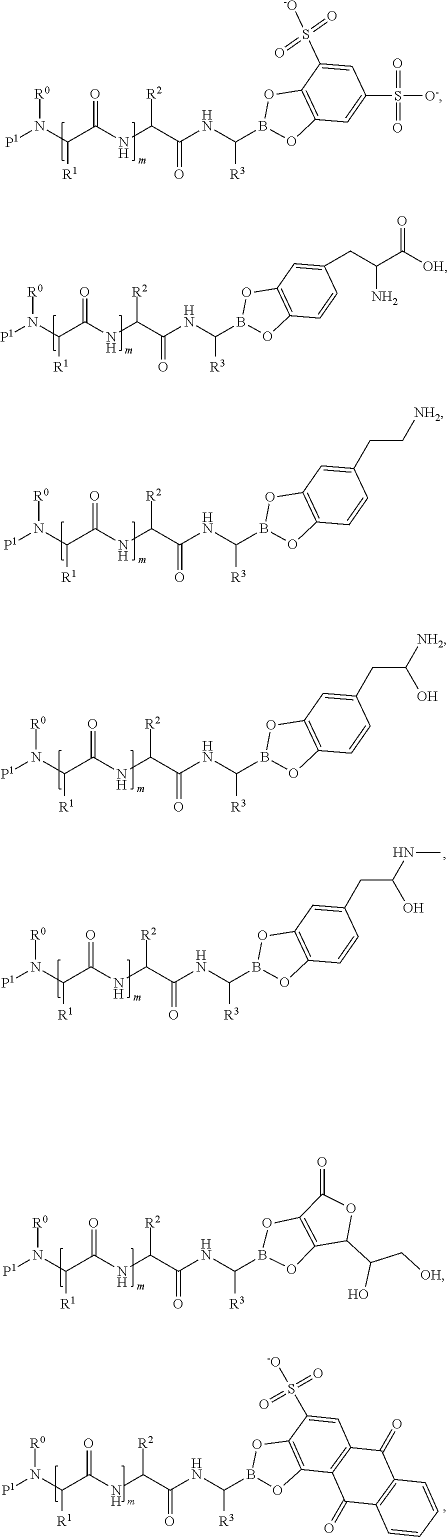

or a pharmaceutically acceptable salt.

In certain examples, the capturing agent-active agent complex is a compound can be defined by Formula IC

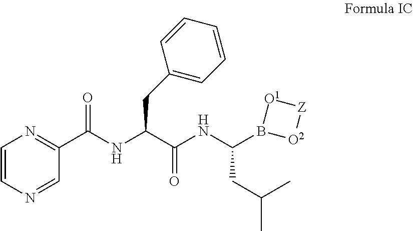

wherein Z, together with O¹ and O², represent a capture agent, wherein Z comprises one or more charged moieties as defined above for Formula I, or
a pharmaceutically acceptable salt thereof.

In some embodiments, the one or more charged moieties can comprise an anionic moiety (e.g., from one to three anionic moieties). In some embodiments, the one or more charged moieties can comprise a cationic moiety (e.g., from one to three cationic moieties). In some embodiments, the one or more charged moieties can comprise one or more anionic moieties and one or more cationic moieties.

In certain examples, the capturing agent-active agent complex is compound can be defined by Formula ID

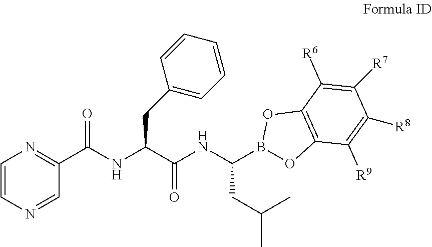

wherein
R⁶-R⁹ are independently hydrogen, oxo, hydroxy, carboxylic acid, carboxylate, sulfonic acid, sulfonate, phosphoryl group, a primary amine, a secondary amine, a tertiary amine, quaternary amine, substituted or unsubstituted alkyl group, cycloalkyl group, heterocyclyl group, aryl group, heteroaryl group, alkylaryl group, arylalkyl group, alkylcycloalkyl group, alkylheterocyclyl group, alkylheteroaryl group, alkoxy group, alkylthio group, or alkylamino group; and at least one of R⁶-R⁹ is a charged moiety; or
a pharmaceutically acceptable salt thereof.

In some embodiments, each charged moiety can comprise an anionic moiety. In some embodiments, each charged moiety can comprise a cationic moiety. In some embodiments, at least one of R⁶-R⁹ can comprise an anionic moiety and at least one of R⁶-R⁹ can comprise a cationic moiety.

In certain examples, the capturing agent-active agent complex is a compound defined by Formula IE:

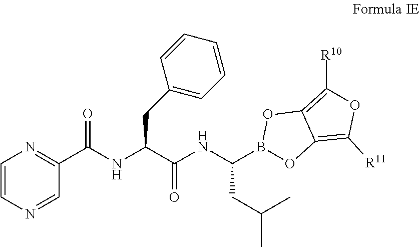

wherein
R¹⁰ and R¹¹ are independently hydrogen, oxo, hydroxy, carboxylic acid, carboxylate, sulfonic acid, sulfonate, phosphoryl group, a primary amine, a secondary amine, a tertiary amine, quaternary amine, substituted or unsubstituted alkyl group, cycloalkyl group, heterocyclyl group, aryl group, heteroaryl group, alkylaryl group, arylalkyl group, alkylcycloalkyl group, alkylheterocyclyl group, alkylheteroaryl group, alkoxy group, alkylthio group, or alkylamino group; and at least one of R¹⁰ or R¹¹ is a charged moiety; or
a pharmaceutically acceptable salt thereof.

In some embodiments, each charged moiety can comprise an anionic moiety. In some embodiments, each charged moiety can comprise a cationic moiety

In some embodiments, the capturing agent-active agent complex is a compound defined by Formula IF:

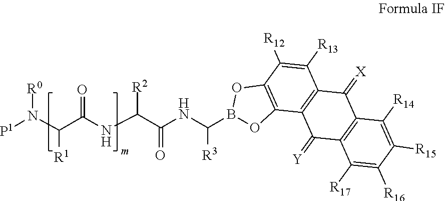

wherein
P¹ is hydrogen or an amino-group protecting moiety;
R⁰ is hydrogen or an alkyl group;
R¹, R², and R³ are independently hydrogen, an alkyl group, a cycloalkyl group, a heterocyclyl group, an aryl group, a heteroaryl group, or —CH₂—R⁴;
m is 0, 1, or 2;
X and Y are independently N, O, or S;
R¹²-R¹⁷ are independently hydrogen, oxo, hydroxy, carboxylic acid, carboxylate, sulfonic acid, sulfonate, phosphoryl group, a primary amine, a secondary amine, a tertiary amine, quaternary amine, substituted or unsubstituted alkyl group, cycloalkyl group, heterocyclyl group, aryl group, heteroaryl group, alkylaryl group, arylalkyl group, alkylcycloalkyl group, alkylheterocyclyl group, alkylheteroaryl group, alkoxy group, alkylthio group, or alkylamino group; and at least one of R⁶-R⁹ is a charged moiety; or
a pharmaceutically acceptable salt thereof.

In some embodiments, each charged moiety can comprise an anionic moiety. In some embodiments, each charged moiety can comprise a cationic moiety. In some embodiments, at least one of R¹²-R¹⁷ can comprise an anionic moiety and at least one of R¹²-R¹⁷ can comprise a cationic moiety.

In some embodiments, the charged moiety can be an anionic moiety. In some other embodiments, the charged moiety can be a cationic moiety. In some other embodiments, the charged moiety can be a zwitterionic moiety.

In some embodiments, the capturing agent-active agent complex is a compound defined by Formula IG:

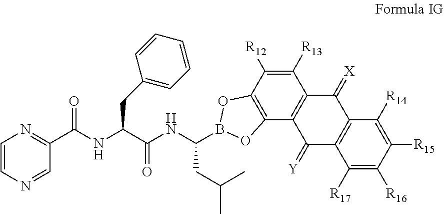

wherein
X and Y are independently N, O, or S;
R¹²-R¹⁷ are independently hydrogen, oxo, hydroxy, carboxylic acid, carboxylate, sulfonic acid, sulfonate, phosphoryl group, a primary amine, a secondary amine, a tertiary amine, quaternary amine, substituted or unsubstituted alkyl group, cycloalkyl group, heterocyclyl group, aryl group, heteroaryl group, alkylaryl group, arylalkyl group, alkylcycloalkyl group, alkylheterocyclyl group, alkylheteroaryl group, alkoxy group, alkylthio group, or alkylamino group; and at least one of R¹²-R¹⁷ is an charged moiety; or
a pharmaceutically acceptable salt thereof.

In some embodiments, each charged moiety can comprise an anionic moiety. In some embodiments, each charged moiety can comprise a cationic moiety. In some embodiments, at least one of R¹²-R¹⁷ can comprise an anionic moiety and at least one of R¹²-R¹⁷ can comprise a cationic moiety.

In some embodiments, the capturing agent-active agent complex can be a compound defined by Formula IH:

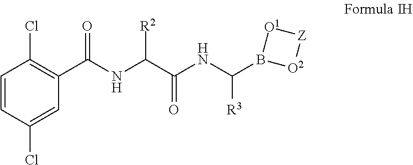

wherein
Z, together with O¹ and O², represent a capture agent, wherein Z comprises one or more charged moieties;
R² and R³ are independently hydrogen, an alkyl group, a cycloalkyl group, a heterocyclyl group, an aryl group, a heteroaryl group, an alkoxy, or —CH₂—R⁴; and
R⁴ is an aryl group, an alkylaryl group, an arylalkyl group, a cycloalkyl group, an alkylcycloalkyl group, a heterocyclyl group, an alkylheterocyclyl group, a heteroaryl group, an alkylheteroaryl group, an alkoxy group, or an alkylthio group;
or a pharmaceutically acceptable salt thereof.

In some embodiments, the capturing agent-active agent complex is a compound defined by Formula IJ:

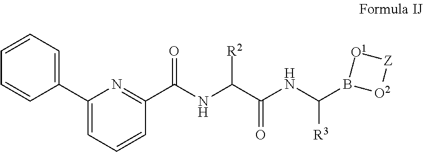

wherein
Z, together with O¹ and O², represent a capture agent, wherein Z comprises one or more charged moieties;
R² and R³ are independently hydrogen, an alkyl group, a cycloalkyl group, a heterocyclyl group, an aryl group, a heteroaryl group, an alkoxy, or —CH₂—R⁴; and
R⁴ is an aryl group, an alkylaryl group, an arylalkyl group, a cycloalkyl group, an alkylcycloalkyl group, a heterocyclyl group, an alkylheterocyclyl group, a heteroaryl group, an alkylheteroaryl group, an alkoxy group, or an alkylthio group;
or a pharmaceutically acceptable salt thereof.

In certain examples, the capturing agent-active agent complex is a compound as follows:

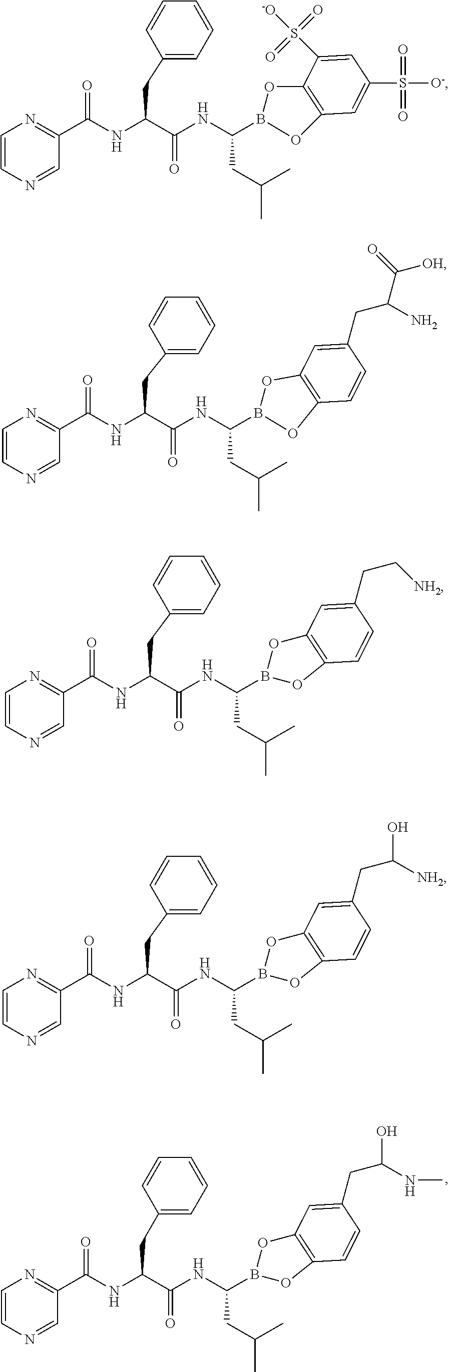

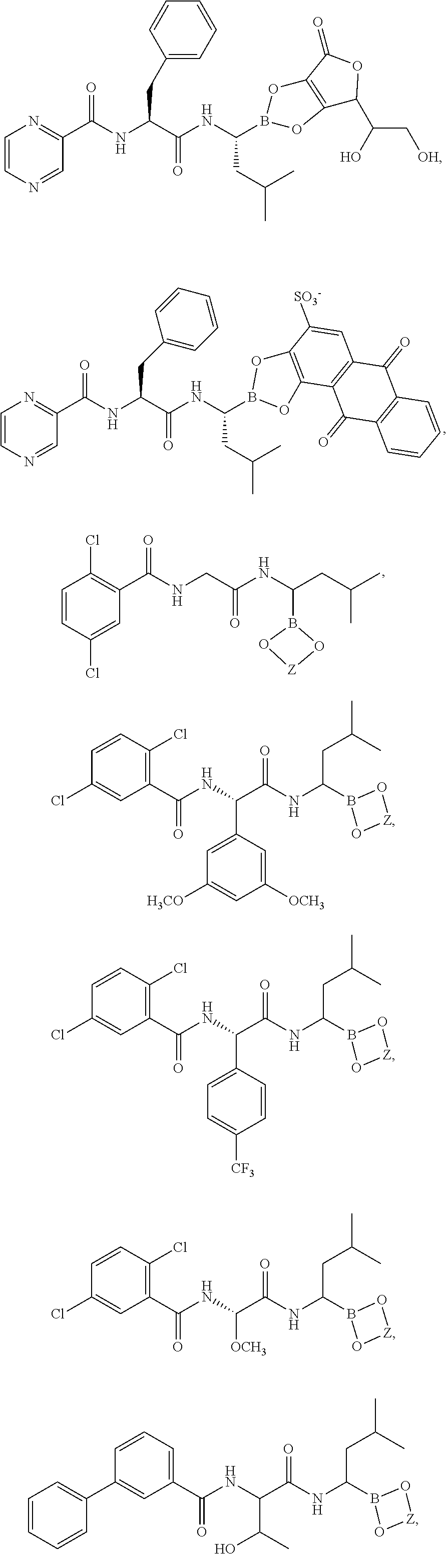

or a pharmaceutically acceptable salt thereof.

In certain examples, the capturing agent-active agent complex is a compound as follows:

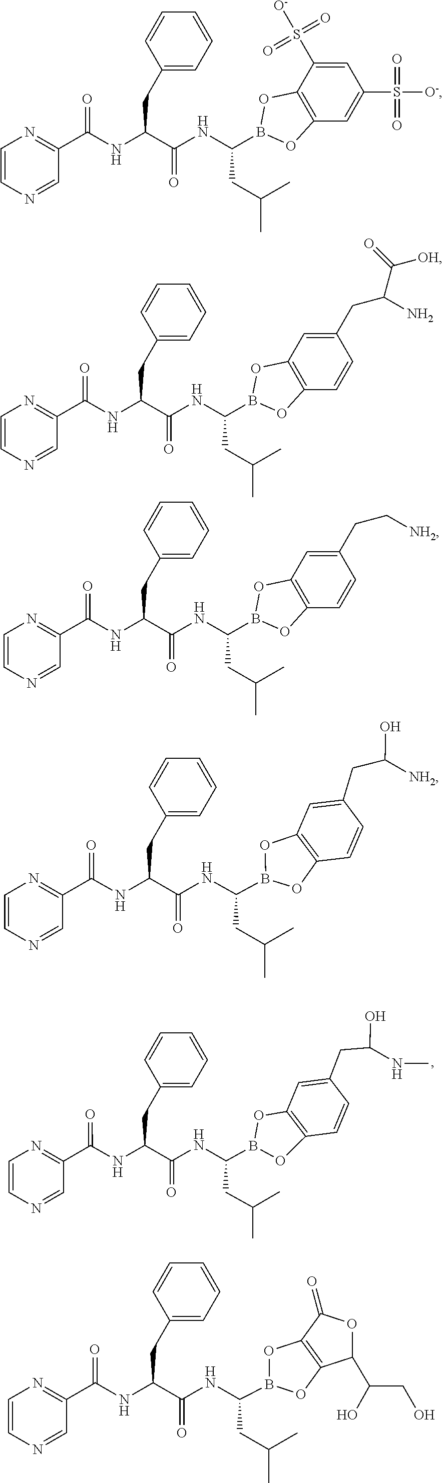

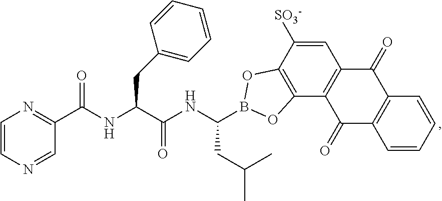

or a pharmaceutically acceptable salt thereof.

In certain examples, the capturing agent-active agent complex is a compound as follows:

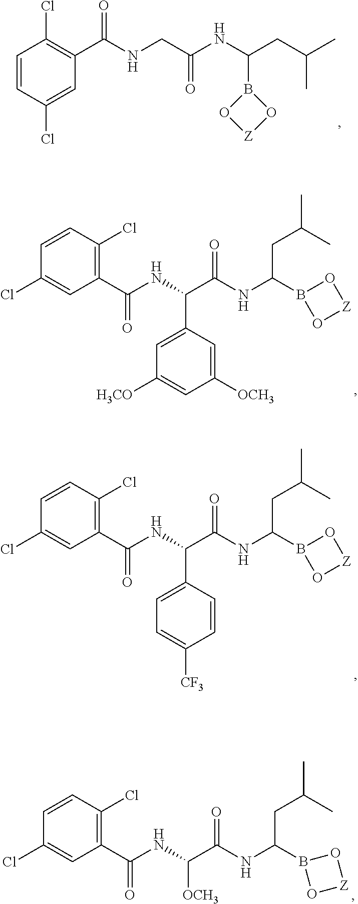

or a pharmaceutically acceptable salt thereof.

In certain examples, the capturing agent-active agent complex is a compound as follows:

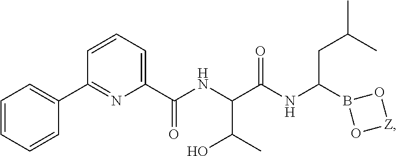

or a pharmaceutically acceptable salt thereof.

### Methods of Treatment

Methods of treating a clinical condition by administration of a disclosed formulation are also provided herein. A clinical condition can be a clinical disorder, disease, dysfunction or other condition that can be ameliorated by a therapeutic composition. In one aspect, the present disclosure provides methods for treating cancer in a subject in need thereof, wherein the method comprises administering a therapeutically effective amount of a liposomal formulation described herein.

The term “neoplasia” or “cancer” is used throughout this disclosure to refer to the pathological process that results in the formation and growth of a cancerous or malignant neoplasm, i.e., abnormal tissue (solid) or cells (non-solid) that grow by cellular proliferation, often more rapidly than normal and continues to grow after the stimuli that initiated the new growth cease. Malignant neoplasms show partial or complete lack of structural organization and functional coordination with the normal tissue and most invade surrounding tissues, can metastasize to several sites, are likely to recur after attempted removal and may cause the death of the patient unless adequately treated. As used herein, the term neoplasia is used to describe all cancerous disease states and embraces or encompasses the pathological process associated with malignant, hematogenous, ascitic and solid tumors. The cancers which may be treated by the compositions disclosed herein may comprise carcinomas, sarcomas, lymphomas, leukemias, germ cell tumors, or blastomas.

Carcinomas which may be treated by the compositions of the present disclosure include, but are not limited to, acinar carcinoma, acinous carcinoma, alveolar adenocarcinoma, carcinoma adenomatosum, adenocarcinoma, carcinoma of adrenal cortex, alveolar carcinoma, alveolar cell carcinoma, basal cell carcinoma, carcinoma basocellular, basaloid carcinoma, basosquamous cell carcinoma, breast carcinoma, bronchioalveolar carcinoma, bronchiolar carcinoma, cerebriform carcinoma, cholangiocellular carcinoma, chorionic carcinoma, colloid carcinoma, comedocarcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epibulbar carcinoma, epidermoid carcinoma, carcinoma epitheliate adenoids, carcinoma exulcere, carcinoma fibrosum, gelatinform carcinoma, gelatinous carcinoma, giant cell carcinoma, gigantocellulare, glandular carcinoma, granulose cell carcinoma, hair matrix carcinoma, hematoid carcinoma, hepatocellular carcinoma, Hurthle cell carcinoma,hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, lentivular carcinoma, carcinoma lenticulare, lipomatous carcinoma, lymphoepithelial carcinoma, carcinoma mastotoids, carcinoma medullare, medullary carcinoma, carcinoma melanodes, melanotonic carcinoma, mucinous carcinoma, carcinoma muciparum, carcinoma mucocullare, mucoepidermoid carcinoma, mucous carcinoma, carcinoma myxomatodes, masopharyngeal carcinoma, carcinoma nigrum, oat cell carcinoma, carcinoma ossificans, osteroid carcinoma, ovarian carcinoma, papillary carcinoma, periportal carcinoma, preinvasive carcinoma, prostate carcinoma, renal cell carcinoma of kidney, reserve cell carcinoma, carcinoma sarcomatodes, scheinderian carcinoma, scirrhous carcinoma, carcinoma scrota, signet-ring cell carcinoma, carcinoma simplex, small cell carcinoma, solandoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberrosum, tuberous carcinoma, verrucous carcinoma, and carcinoma vilosum.

Representative sarcomas which may be treated by the compositions of the present disclosure include, but are not limited to, liposarcomas (including myxoid liposarcomas and pleomorphic liposarcomas), leiomyosarcomas, rhabdomyosarcomas, neurofibrosarcomas, malignant peripheral nerve sheath tumors, Ewing's tumors (including Ewing's sarcoma of bone, extraskeletal or non-bone) and primitive neuroectodermal tumors (PNET), synovial sarcoma, hemangioendothelioma, fibrosarcoma, desmoids tumors, dermatofibrosarcoma protuberance (DFSP), malignant fibrous hi stiocytoma(MFH), hemangiopericytoma, malignant mesenchymoma, alveolar soft-part sarcoma, epithelioid sarcoma, clear cell sarcoma, desmoplastic small cell tumor, gastrointestinal stromal tumor (GIST) and osteosarcoma (also known as osteogenic sarcoma) skeletal and extra-skeletal, and chondrosarcoma.

The compositions of the present disclosure may be used in the treatment of a lymphoma. Lymphomas which may be treated include mature B cell neoplasms, mature T cell and natural killer (NK) cell neoplasms, precursor lymphoid neoplasms, Hodgkin lymphomas, and immunodeficiency-associated lymphoproliferative disorders.

Representative mature B cell neoplasms include, but are not limited to, B-cell chronic lymphocytic leukemia/small cell lymphoma, B-cell prolymphocytic leukemia, lymphoplasmacytic lymphoma (such as Waldenström macroglobulinemia), splenic marginal zone lymphoma, hairy cell leukemia, plasma cell neoplasms (such as plasma cell myeloma/multiple myeloma, plasmacytoma, monoclonal immunoglobulin deposition diseases, and heavy chain diseases), extranodal marginal zone B cell lymphoma (MALT lymphoma), nodal marginal zone B cell lymphoma, follicular lymphoma, primary cutaneous follicular center lymphoma, mantle cell lymphoma, diffuse large B cell lymphoma, diffuse large B-cell lymphoma associated with chronic inflammation, Epstein-Barr virus-positive DLBCL of the elderly, lymphomatoid granulomatosis, primary mediastinal (thymic) large B-cell lymphoma, intravascular large B-cell lymphoma, ALK+ large B-cell lymphoma, plasmablastic lymphoma, primary effusion lymphoma, large B-cell lymphoma arising in HHV8-associated multicentric Castleman's disease, and Burkitt lymphoma/leukemia. Representative mature T cell and NK cell neoplasms include, but are not limited to, T-cell prolymphocytic leukemia, T-cell large granular lymphocyte leukemia, aggressive NK cell leukemia, adult T-cell leukemia/lymphoma, extranodal NK/T-cell lymphoma, nasal type, enteropathy-associated T-cell lymphoma, hepatosplenic T-cell lymphoma, blastic NK cell lymphoma, lycosis fungoides/Sezary syndrome, primary cutaneous CD30-positive T cell lymphoproliferative disorders (such as primary cutaneous anaplastic large cell lymphoma and lymphomatoid papulosis), peripheral T-cell lymphoma not otherwise specified, angioimmunoblastic T cell lymphoma, and anaplastic large cell lymphoma. Representative precursor lymphoid neoplasms include B-lymphoblastic leukemia/lymphoma not otherwise specified, B-lymphoblastic leukemia/lymphoma with recurrent genetic abnormalities, or T-lymphoblastic leukemia/lymphoma. Representative Hodgkin lymphomas include classical Hodgkin lymphomas, mixed cellularity Hodgkin lymphoma, lymphocyte-rich Hodgkin lymphoma, and nodular lymphocyte-predominant Hodgkin lymphoma.

The compositions of the present disclosure may be used in the treatment of a Leukemia. Representative examples of leukemias include, but are not limited to, acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia, adult T-cell leukemia, clonal eosinophilias, and transient myeloproliferative disease.

The compositions of the present disclosure may be used in the treatment of a germ cell tumor, for example germinomatous (such as germinoma, dysgerminoma, and seminoma), non germinomatous (such as embryonal carcinoma, endodermal sinus tumor, choriocarcinoma, teratoma, polyembryoma, and gonadoblastoma) and mixed tumors.

The compositions of the present disclosure may be used in the treatment of blastomas, for example hepatoblastoma, medulloblastoma, nephroblastoma, neuroblastoma, pancreatoblastoma, pleuropulmonary blastoma, retinoblastoma, and glioblastoma multiforme.

Representative cancers which may be treated include, but are not limited to: bone and muscle sarcomas such as chondrosarcoma, Ewing's sarcoma, malignant fibrous histiocytoma of bone/osteosarcoma, osteosarcoma, rhabdomyosarcoma, and heart cancer; brain and nervous system cancers such as astrocytoma, brainstem glioma, pilocytic astrocytoma, ependymoma, primitive neuroectodermal tumor, cerebellar astrocytoma, cerebral astrocytoma, glioma, medulloblastoma, neuroblastoma, oligodendroglioma, pineal astrocytoma, pituitary adenoma, and visual pathway and hypothalamic glioma; breast cancers including invasive lobular carcinoma, tubular carcinoma, invasive cribriform carcinoma, medullary carcinoma, male breast cancer, Phyllodes tumor, and inflammatory breast cancer; endocrine system cancers such as adrenocortical carcinoma, islet cell carcinoma, multiple endocrine neoplasia syndrome, parathyroid cancer, phemochromocytoma, thyroid cancer, and Merkel cell carcinoma; eye cancers including uveal melanoma and retinoblastoma; gastrointestinal cancers such as anal cancer, appendix cancer, cholangiocarcinoma, gastrointestinal carcinoid tumors, colon cancer, extrahepatic bile duct cancer, gallbladder cancer, gastric cancer, gastrointestinal stromal tumor, hepatocellular cancer, pancreatic cancer, and rectal cancer; genitourinary and gynecologic cancers such as bladder cancer, cervical cancer, endometrial cancer, extragonadal germ cell tumor, ovarian cancer, ovarian epithelial cancer, ovarian germ cell tumor, penile cancer, renal cell carcinoma, renal pelvis and ureter transitional cell cancer, prostate cancer, testicular cancer, gestational trophoblastic tumor, urethral cancer, uterine sarcoma, vaginal cancer, vulvar cancer, and Wilms tumor; head and neck cancers such as esophageal cancer, head and neck cancer, nasopharyngeal carcinoma, oral cancer, oropharyngeal cancer, paranasal sinus and nasal cavity cancer, pharyngeal cancer, salivary gland cancer, and hypopharyngeal cancer; hematopoietic cancers such as acute biphenotypic leukemia, acute eosinophilic leukemia, acute lymphoblastic leukemia, acute myeloid leukemia, acute myeloid dendritic cell leukemia, AIDS-related lymphoma, anaplastic large cell lymphoma, angioimmunoblastic T-cell lymphoma, B-cell prolymphocytic leukemia, Burkitt's lymphoma, chronic lymphocytic leukemia, chronic myelogenous leukemia, cutaneous T-cell lymphoma, diffuse large B-cell lymphoma, follicular lymphoma, hairy cell leukemia, hepatosplenic T-cell lymphoma, Hodgkin's lymphoma, hairy cell leukemia, intravascular large B-cell lymphoma, large granular lymphocytic leukemia, lymphoplasmacytic lymphoma, lymphomatoid granulomatosis, mantle cell lymphoma, marginal zone B-cell lymphoma, Mast cell leukemia, mediastinal large B cell lymphoma, multiple myeloma/plasma cell neoplasm, myelodysplastic syndromes, mucosa-associated lymphoid tissue lymphoma, mycosis fungoides, nodal marginal zone B cell lymphoma, non-Hodgkin lymphoma, precursor B lymphoblastic leukemia, primary central nervous system lymphoma, primary cutaneous follicular lymphoma, primary cutaneous immunocytoma, primary effusion lymphoma, plasmablastic lymphoma, Sezary syndrome, splenic marginal zone lymphoma, and T-cell prolymphocytic leukemia; skin cancers such as basal cell carcinoma, squamous cell carcinoma, skin adnexal tumors (such as sebaceous carcinoma), melanoma, Merkel cell carcinoma, sarcomas of primary cutaneous origin (such as dermatofibrosarcoma protuberans), and lymphomas of primary cutaneous origin (such as mycosis fungoides); thoracic and respiratory cancers such as bronchial adenomas/carcinoids, small cell lung cancer, mesothelioma, non-small cell lung cancer, pleuropulmonary blastoma, laryngeal cancer, and thymoma or thymic carcinoma; HIV/AIDs-related cancers such as Kaposi sarcoma; epithelioid hemangioendothelioma; desmoplastic small round cell tumor; and liposarcoma.

### Methods of Administration

The formulations as used in the methods described herein can be administered by any suitable method and technique presently or prospectively known to those skilled in the art. For example, the active components described herein can be formulated in a physiologically-or pharmaceutically-acceptable form and administered by any suitable route known in the art including, for example, oral and parenteral routes of administering. As used herein, the term “parenteral” includes subcutaneous, intradermal, intravenous, intramuscular, intraperitoneal, and intrasternal administration, such as by injection. Administration of the active components of their compositions can be a single administration, or at continuous and distinct intervals as can be readily determined by a person skilled in the art.

Formulations, as described herein, can include one or more pharmaceutically acceptable excipients.

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include, but are not limited to, any non-toxic, inert solid, semisolid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition or cosmetic composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray. In some embodiments, the active compounds disclosed herein are administered topically.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chl orhexi dine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, various gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacrylic acid/acrylamide copolymer, polyalkylene oxides such as polyethylene oxide, polypropylene oxide, poly(ethylene oxide-propylene oxide), and a Pluronic polymer, polyoxy ethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy(Polyethylene glycol)-1000], 1,2-Distearoyl-sn-glycero-3-Phosphoethanolamine-N-[Methoxy(Polyethylene glycol)-2000], and 1,2-Distearoyl-sn-glycero-3-Phosphoethanolamine-N-[Methoxy(Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly (meth) acrylic acid, and esters amide and hydroxy alkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain embodiments, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be an injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain embodiments, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration may be in the form of suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the particles.

Formulations disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and formulations disclosed herein can be systemically administered, such as intravenously or orally, optionally in combination with a pharmaceutically acceptable carrier such as an inert diluent, or an assimilable edible carrier for oral delivery. They can be enclosed in hard or soft shell gelatin capsules, can be compressed into tablets, or can be incorporated directly with the food of the patient's diet. For oral therapeutic administration, the active compound can be combined with one or more excipients and used in the form of ingestible tablets, buccal tablets, troches, capsules, elixirs, suspensions, syrups, wafers, aerosol sprays, and the like.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard- filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard- filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate controlling membrane or by dispersing the particles in a polymer matrix or gel.

The active ingredient may be administered in such amounts, time, and route deemed necessary in order to achieve the desired result. The exact amount of the active ingredient will vary from subject to subject, depending on the species, age, and general condition of the subject, the severity of the infection, the particular active ingredient, its mode of administration, its mode of activity, and the like. The active ingredient, whether the active compound itself, or the active compound in combination with an agent, is preferably formulated in dosage unit form for ease of administration and uniformity of dosage. It will be understood, however, that the total daily usage of the active ingredient will be decided by the attending physician within the scope of sound medical judgment. The specific therapeutically effective dose level for any particular subject will depend upon a variety of factors including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The active ingredient may be administered by any route. In some embodiments, the active ingredient is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the active ingredient (e.g., its stability in the environment of the gastrointestinal tract), the condition of the subject (e.g., whether the subject is able to tolerate oral administration), etc.

The exact amount of an active ingredient required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

Useful dosages of the compounds and agents and pharmaceutical formulations disclosed herein can be determined by comparing their in vitro activity, and in vivo activity in animal models. Methods for the extrapolation of effective dosages in mice, and other animals, to humans are known to the art; for example, see U.S. Pat. No. 4,938,949.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

For the treatment of oncological disorders, the compounds disclosed herein can be administered to a patient in need of treatment in combination with other antitumor or anticancer substances and/or with radiation and/or photodynamic therapy and/or with surgical treatment to remove a tumor. These other substances or treatments can be given at the same as or at different times from the compounds disclosed herein. For example, the compounds disclosed herein can be used in combination with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation) and HERCEPTIN (Genentech, Inc.), respectively.

Many tumors and cancers have viral genome present in the tumor or cancer cells. For example, Epstein-Barr Virus (EBV) is associated with a number of mammalian malignancies. The compounds disclosed herein can also be used alone or in combination with anticancer or antiviral agents, such as ganciclovir, azidothymidine (AZT), lamivudine (3TC), etc., to treat patients infected with a virus that can cause cellular transformation and/or to treat patients having a tumor or cancer that is associated with the presence of viral genome in the cells. The compounds disclosed herein can also be used in combination with viral based treatments of oncologic disease. For example, the compounds can be used with mutant herpes simplex virus in the treatment of non-small cell lung cancer (Toyoizumi, et al., “Combined therapy with chemotherapeutic agents and herpes simplex virus type IICP34.5 mutant (HSV-1716) in human non-small cell lung cancer,” Human Gene Therapy, 1999, 10(18):17).

For the treatment of oncological disorders, compounds and agents and compositions disclosed herein can be administered to a patient in need of treatment prior to, subsequent to, or in combination with other antitumor or anticancer agents or substances (e.g., chemotherapeutic agents, immunotherapeutic agents, radiotherapeutic agents, cytotoxic agents, etc.) and/or with radiation therapy and/or with surgical treatment to remove a tumor. For example, compounds and agents and compositions disclosed herein can be used in methods of treating cancer wherein the patient is to be treated or is or has been treated with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation; East Hanover, NJ) and HERCEPTIN (Genentech, Inc.; South San Francisco, CA), respectively. These other substances or radiation treatments can be given at the same as or at different times from the compounds disclosed herein. Examples of other suitable chemotherapeutic agents include, but are not limited to, altretamine, bleomycin, bortezomib (VELCADE), busulphan, calcium folinate, capecitabine, carboplatin, carmustine, chlorambucil, cisplatin, cladribine, crisantaspase, cyclophosphamide, cytarabine, dacarbazine, dactinomycin, daunorubicin, docetaxel, doxorubicin, epirubicin, etoposide, fludarabine, fluorouracil, gefitinib (IRESSA), gemcitabine, hydroxyurea, idarubicin, ifosfamide, imatinib (GLEEVEC), irinotecan, liposomal doxorubicin, lomustine, melphalan, mercaptopurine, methotrexate, mitomycin, mitoxantrone, oxaliplatin, paclitaxel, pentostatin, procarbazine, raltitrexed, streptozocin, tegafur-uracil, temozolomide, thiotepa, tioguanine/thioguanine, topotecan, treosulfan, vinblastine, vincristine, vindesine, vinorelbine. In an exemplified embodiment, the chemotherapeutic agent is melphalan. Examples of suitable immunotherapeutic agents include, but are not limited to, alemtuzumab, cetuximab (ERBITUX), gemtuzumab, iodine 131 tositumomab, rituximab, trastuzamab (HERCEPTIN). Cytotoxic agents include, for example, radioactive isotopes (e.g., I¹³¹, I¹²⁵, Y⁹⁰, P³², etc.), and toxins of bacterial, fungal, plant, or animal origin (e.g., ricin, botulinum toxin, anthrax toxin, aflatoxin, jellyfish venoms (e.g., box jellyfish, etc.) Also disclosed are methods for treating an oncological disorder comprising administering an effective amount of a compound and/or agent disclosed herein prior to, subsequent to, and/or in combination with administration of a chemotherapeutic agent, an immunotherapeutic agent, a radiotherapeutic agent, or radiotherapy.

### Kits

The disclosed subject matter also concerns a packaged dosage formulation comprising in one or more containers at least one inhibitor compound or composition disclosed herein, e.g., any compound of Formula I. A packaged dosage formulation can optionally comprise in one or more containers a pharmaceutically acceptable carrier or diluent.

Depending upon the disorder or disease condition to be treated, a suitable dose(s) can be that amount that will reduce proliferation or growth of the target cell(s). In the context of cancer, a suitable dose(s) is that which will result in a concentration of the active agent in cancer tissue, such as a malignant tumor, which is known to achieve the desired response. The preferred dosage is the amount which results in maximum inhibition of cancer cell growth, without unmanageable side effects. Administration of a compound and/or agent can be continuous or at distinct intervals, as can be determined by a person of ordinary skill in the art.

To provide for the administration of such dosages for the desired therapeutic treatment, in some embodiments, pharmaceutical compositions disclosed herein can comprise between about 0.1% and 45%, and especially, 1 and 15%, by weight of the total of one or more of the compounds based on the weight of the total composition including carrier or diluents. Illustratively, dosage levels of the administered active ingredients can be: intravenous, 0.01 to about 20 mg/kg; intraperitoneal, 0.01 to about 100 mg/kg; subcutaneous, 0.01 to about 100 mg/kg; intramuscular, 0.01 to about 100 mg/kg; oral, 0.01 to about 100 mg/kg; of animal (body) weight. In some embodiments, the dosage levels of the administered active ingredients can be: intravenous, 0.01 to about 2.5 mg/kg; intraperitoneal, 0.01 to about 5 mg/kg; subcutaneous, 0.01 to about 5 mg/kg; intramuscular, 0.01 to about 5 mg/kg; oral, 0.01 to about 5 mg/kg; of animal (body) weight. In some embodiments, the dosage levels of the administered active ingredients can be: intravenous, 0.01 to about 1 mg/kg; intraperitoneal, 0.01 to about 5 mg/kg; subcutaneous, 0.01 to about 5 mg/kg; intramuscular, 0.01 to about 5 mg/kg; oral, 0.01 to about 5 mg/kg; of animal (body) weight. In certain embodiments, the dosage levels of the administered active ingredients can be: intravenous, 0.01 to about 0.5 mg/kg; intraperitoneal, 0.01 to about 1 mg/kg; subcutaneous, 0.01 to about 1 mg/kg; intramuscular, 0.01 to about 1 mg/kg; oral, 0.01 to about 1 mg/kg; of animal (body) weight.

Also disclosed are kits that comprise a composition comprising a compound disclosed herein in one or more containers. The disclosed kits can optionally include pharmaceutically acceptable carriers and/or diluents. In one embodiment, a kit includes one or more other components, adjuncts, or adjuvants as described herein. In another embodiment, a kit includes one or more anti-cancer agents, such as those agents described herein. In one embodiment, a kit includes instructions or packaging materials that describe how to administer a compound or composition of the kit. Containers of the kit can be of any suitable material, e.g., glass, plastic, metal, etc., and of any suitable size, shape, or configuration. In one embodiment, a compound and/or agent disclosed herein is provided in the kit as a solid, such as a tablet, pill, or powder form. In another embodiment, a compound and/or agent disclosed herein is provided in the kit as a liquid or solution. In one embodiment, the kit comprises an ampoule or syringe containing a compound and/or agent disclosed herein in liquid or solution form.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

Formulation disclosed here represents a significant improvement over previously synthesized meglumine lipophilic derivatives for BTZ in terms of the stability of the liposomal formulation and, therefore, has much greater potential for clinical translation. Potential applications would be in therapy of hemoatologic malignancies (MM, MCL, AML, and other leukemia and lymphomas) as well as solid tumors. The advantage would be reduced toxicity and increased efficacy enabling improved therapeutic response.

Ascorbic acid (ASC) (vitamin C) is a water-soluble vitamin. ASC is known to form a stable complex with boric acid via its planar diol. There is anecdotal evidence that ascorbic acid forms a stable complex with BTZ. In the current invention, a series of stable liposomal formulations of BTZ are disclosed based on using ASC or its derivatives, such as L-ascorbyl-6-palmitate as BTZ binding agent. The described L- BTZ formulations are highly stable and are based on excipients that are readily available and have previous history of safe human use and generally regarded as safe (GRAS). The liposomes can be loaded with BTZ using a remote loading procedure using the ASC or ASC derivative as a BTZ binding agent.

Alternatively, BTZ can be pre-complex to an ASC derivative to form a lipophilic prodrug and then incorporated into liposomes. The liposomes (L-BTZ) can be lyophilized in the presence of a lyoprotectant such as trehalose and sucrose to form a dry cake for longer term stability. The key advantage of the novel formulations is that the ASC-BTZ is quite stable and yet still capable of releasing the BTZ at an adequate rate under physiological conditions. Therefore, the L-BTZ has the optimal stability profile to enable clinical utility of this formulation.

Important to the BTZ loading into liposomes is the formation of a meta-stable boronic ester with a ligand that contains planar diols and alkaline intraliposomal pH. High intraliposomal pH can be created by making liposomes in the presence of pH 8 (7-9) sodium phosphate buffer (e.g., 50-100 mM) or another buffer (e.g., HEPES buffer) followed by removal of external buffer by gel filtration, dialysis or diafiltration.

Elevated pH can also be obtained by making liposomes in the presence of Ca acetate, sodium acetate, or sodium carbonate. Following removal of external buffer, the selective release of free acid creates high pH condition inside the liposomes. Both approaches can be used to create a high pH intraliposomal environment that facilitates drug retention.

### Example 1

Remote loading of BTZ into liposomes encapsulating calcium ascorbate/calcium acetate. Lipids (DSPC/Chol/mPEG-DSPE 60:35:5 mole/mole) were dissolved in chloroform, dried into a thin film on a round-bottom flask and hydrated in 100 mM calcium ascorbate/50 mM calcium acetate (pH 8). The resulting liposomes were subjected to 5 cycles of freezing and thawing and extrusion through a 100-nm pore size polycarbonate membrane in a Lipex extruder at 65 degree. This resulted in liposomes with a mean diameter of 110 nm with narrow size distribution. External buffer of the liposomes was exchanged to 130 mM NaCl/10 mM histidine (pH 6) by passing through a Sepharose CL-4B column. This resulted in liposomes with an inside high outside low pH gradient. BTZ was dissolved in a small volume of DMSO and added to the liposomes and incubated under stirring at 20-60 degrees for 10-60 min (e.g., 30 min at 50 degree) to facilitate remote loading. BTZ diffuses into the liposomes and forms a complex with ascorbic acid (BTZ-ASC complex), which is negatively charged and partially precipitates intraliposome with calcium, resulting in stable encapsulation of the BTZ. Finally, unencapsulated BTZ was removed from the liposomes by passing through a Sepharose CL-4B column. BTZ concentration in the L-BTZ was determined by UV absorption or by HPLC. A loading efficiency of 60-99% was achieved (e.g., 89%). The liposomes remain stable with no significant changes in particle size and drug encapsulation under storage at 4 degree for 7 days. Less than 5% of BTZ was released based on Sepharose CL-4B column re-analysis of the liposomal sample. For longer term stability the liposomes need to be lyophilized in the presence of 8% trehalose as a lyoprotectant using a 2-stage program.

100 mM calcium ascorbate/50 mM calcium acetate can be replaced with 50 mM sodium ascorbate/100 mM sodium phosphate (pH 8) to maintain intraliposomal pH and the transmembrane pH gradient during lyophilization. The phosphate has buffering capacity that will maintain stability of the L-BTZ better during lyophilization.

### Example 2

Remote loading of BTZ into liposomes containing ascorbyl palmitate (ASC-C16) and encapsulating calcium acetate. Lipids (DSPC/Chol/ASC-C16/mPEG-DSPE 50:25:20:5 mole/mole) were dissolved in chloroform, dried into a thin film on a round-bottom flask and hydrated in 150 mM calcium acetate (pH 8). The resulting liposomes were subjected to 5 cycles of freezing and thawing and extrusion through a 100-nm pore size polycarbonate membrane in a Lipex extruder at 65 degree. This resulted in liposomes with a mean diameter of 110 nm with narrow size distribution. External buffer of the liposomes was exchanged to 130 mM NaCl/10 mM histidine (pH 6) by passing through a Sepharose CL-4B column. This resulted in liposomes with an inside high outside low pH gradient. BTZ was dissolved in a small volume of DMSO and added to the liposomes (or BTZ was added as a dry powder under stirring) and incubated under stirring or shaking at 20-60 degrees for 10-60 min (e.g., 30 min at 50 degree) to facilitate remote loading. BTZ diffuses into the liposomes and forms a complex with ascorbic acid (BTZ-ASC-C16 complex), which is negatively charged and partially precipitates intraliposome with calcium, resulting in stable encapsulation of the BTZ. Some of the ASC-C16 in the outer leaflet also can complex to BTZ but with less stability due to the lower external pH. Finally, unencapsulated BTZ was removed from the liposomes by passing through a Sepharose CL-4B column. BTZ concentration in the L-BTZ was determined by UV absorption or by HPLC. A loading efficiency of 50-90% was achieved (e.g., 81.4%). The liposomes remain stable with no significant changes in particle size and drug encapsulation under storage at 4 degree for 7 days. Less than 5% of BTZ was released based on Sepharose CL-4B column re-analysis of the liposomal sample. It is noted that BTZ can also be pre-mixed with 1-2× of ASC-C16 to form a BTZ-ASC-C16 prodrug and incorporated into liposome as a lipid component in the initial step of liposome preparation. The later formation of the inside-outside pH gradient will drive the redistribution of BTZ into the inner leaflet or internal aqueous phase of the liposomal formulation. In another embodiment, the liposomes are made in the absence of a pH gradient. BTZ still can form a relatively stable complex with ASC-C16 and form a lipophilic prodrug which can be incorporated into liposomes at up to 25 mole % of the total lipids, preferably at 20 mole %. Ascorbyl dipalmitate or any other lipophilic derivatives of ascorbic acid at the 6 or 5 position or 5,6 di-substituted derivatives would be effective replacing ASC-C16.

Isoascorbic acid (erythorbic acid) and its derivatives can be used to substitute ascorbic acid and its derivatives without changing the results. For example, calcium erythorbate can be used in place of Calcium Ascorbate for liposomal BTZ preparation described in example 1. Ascorbate-6-phosphate can be used as a trapping agent as well or any ascorbate derivatives at the 6 position.

### Example 3

It is worth noting that other molecules that contain planer diols can be used in BTZ loading into liposomes. L-DOPA, D-DOPA, DL-DOPA, dopamine and their lipophilic derivatives can also be used as BTZ binding agents either as an aqueous phase trapping agent or as a lipophilic complexing agent for BTZ and used in a similar fashion as ASC and ASC-C16. Also effective is fructose-6-phosphate (F-6-P) as an impermeable binding agent for BTZ inside liposomes. Tiron is another BTZ trapping agent that can be used instead of ascorbic acid. Tiron is superior in that it has very low membrane permeability coefficient and therefore can retain BTZ very efficiently. Salicylic acid, sulfosalicytic acid are alternatively as well.

Remote loading of BTZ into liposomes encapsulating tiron (4,5-Dihydroxy-1,3-benzenedisulfonic acid disodium salt)/calcium acetate. Lipids (DSPC/Chol/mPEG-DSPE 60:35:5 mole/mole) were dissolved in chloroform, dried into a thin film on a round-bottom flask and hydrated in 100 mM tiron/50 mM calcium acetate (pH 8). The resulting liposomes were subjected to 5 cycles of freezing and thawing and extrusion through a 100-nm pore size polycarbonate membrane in a Lipex extruder at 65 degree. This resulted in liposomes with a mean diameter of 110 nm with narrow size distribution. External buffer of the liposomes was exchanged to 130 mM NaCl/10 mM histidine (pH 6) by passing through a Sepharose CL-4B column. This resulted in liposomes with an inside high outside low pH gradient. BTZ was dissolved in a small volume of DMSO and added to the liposomes and incubated under stirring at 20-60 degrees for 10-60 min (e.g., 30 min at 50 degree) to facilitate remote loading. BTZ diffuses into the liposomes and forms a complex with tiron (BTZ-Tiron complex), which is negatively charged and partially precipitates intraliposome with calcium, resulting in stable encapsulation of the BTZ. Finally, unencapsulated BTZ was removed from the liposomes by passing through a Sepharose CL-4B column. BTZ concentration in the L-BTZ was determined by UV absorption or by HPLC. A loading efficiency of 60-99% was achieved (e.g., 92%). The liposomes remain stable with no significant changes in particle size and drug encapsulation under storage at 4 degree for 7 days. Less than 5% of BTZ was released based on Sepharose CL-4B column re-analysis of the liposomal sample.

### Example 4

BTZ and ASC-C16 was combined at 1:2 in the presence of 10×TEA in tert-butanol to form BTZ-ASC-C16 complex. This was combined with phospholipids (DPPC/Chol/BTZ-ASC-C16/mPEG-DSPE) 60:15:20:5 mole/mole) were dissolved in tert-butanol, and hydrated in 50 mM sodium phosphate 100 mM NaCl (pH 8). The resulting liposomes were subjected to sonication or homogenization to reduce particle size to <100 nm. This resulted in liposomes with a mean diameter of 80 nm. Free BTZ was removed by passing through a Sepharose CL-4B column against 50 mM sodium phosphate pH 8. Then, 8% of trehalose was added as a lyoprotectant. The liposomes were lyophilized using a 2-stage drying program. BTZ concentration in the L- BTZ was determined by UV absorption or by HPLC. A loading efficiency of 74% was achieved. The liposomes remain stable with no significant changes in particle size and drug encapsulation under storage at 4 degree for 7 days. Less than 5% of BTZ was released based on Sepharose CL-4B column re-analysis of the liposomal sample.

### Example 5

L-BTZ can be prepared by remote loading. First, a complexing agent is passively encapsulated during liposome formation. Then, BTZ is added externally and allowed to diffuse into the liposomes to form a BTZ complex (cyclic boronic ester). A key aspect of the L-BTZ formulation is the stability of encapsulation, which directly impact its storage shelf-life and in vivo pharmacokinetic profile.

### Tiron-BTZ Complexes are More Stable than Other BTZ Complexes

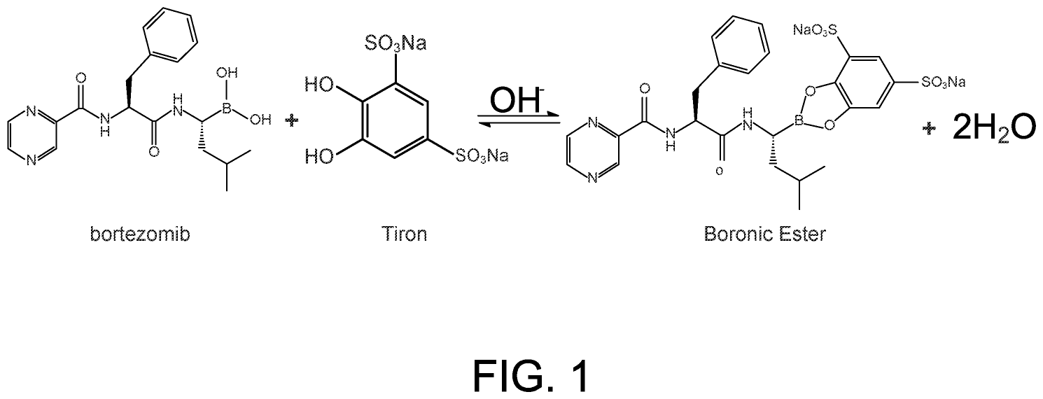

The relative stability of BTZ complexes with diol-containing compounds, which have been used in previously published L-BTZ formulations, was tested. The complex is formed under weekly basic conditions (see FIG. 1 for the reaction involving tiron). Reduction in pH result in reversal of this reaction and regeneration of free BTZ, which is readily extracted into the chloroform phase. Therefore, the pH required for chloroform extraction indicates BTZ complex stability. The lower the pH required, the more stable the BTZ complex. This is highly important to L-BTZ since with tiron, high intraliposomal pH (which limits shelf-life of liposomes) is not required, making it possible to produce a shelf-life and in vivo stability profile acceptable for a drug product.

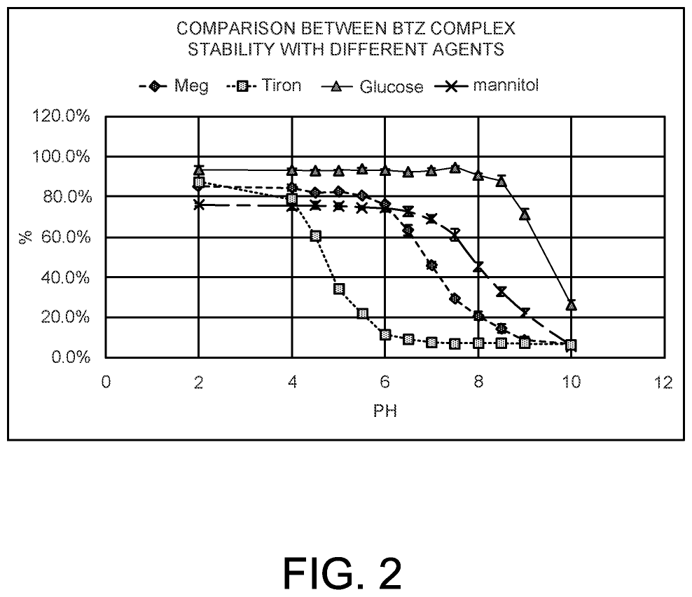

As shown in FIG. 2 the tiron complex showed the highest stability. This was due to the planar configuration of the aromatic catechol, favoring stable boronic ester formulation.

### Preparation of Liposomal Bortezomib

Lipids DSPC/Chol/mPEG-DSPE (60:38:2 mol/mol) were dissolved in ethanol. The lipids solution was injected into Tiron (200 mM)/Sodium acetate (200 mM) solution at 60° C. quickly for rapid hydration. The resulting liposomes were extruded under high pressure through a 100-nm polycarbonate membrane on a Lipex extruder at 60° C. The product was dialyzed against PBS. BTZ was added and the mixture was incubated at room temperature overnight. Unencapsulated BTZ was removed by dialysis against PBS. The product was sterile filtered and stored at 4° C. The liposome product had a mean particle diameter of 115 nm with narrow distribution. The product remained stable at 4° C. for at least 4 months from the time of preparation with <10% drug release during storage.

### Antitumor Activity of L-BTZ in Cell-Derived Multiple Myeloma Murine Xenograft Models and Plasma PK of L-BTZ

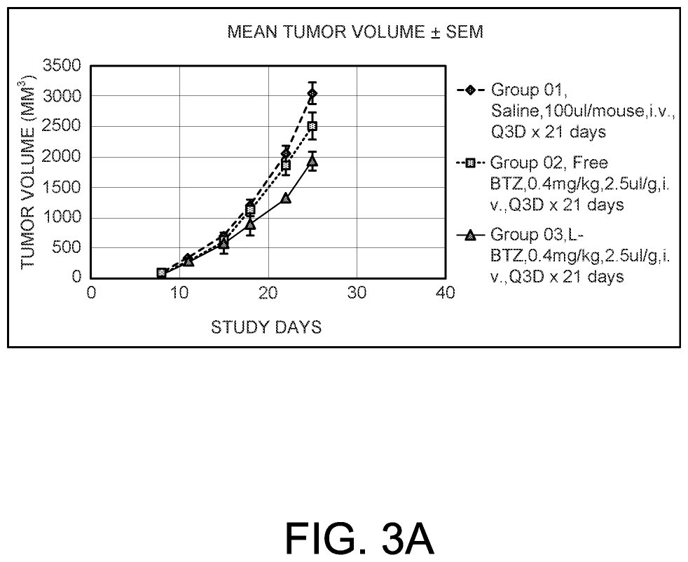

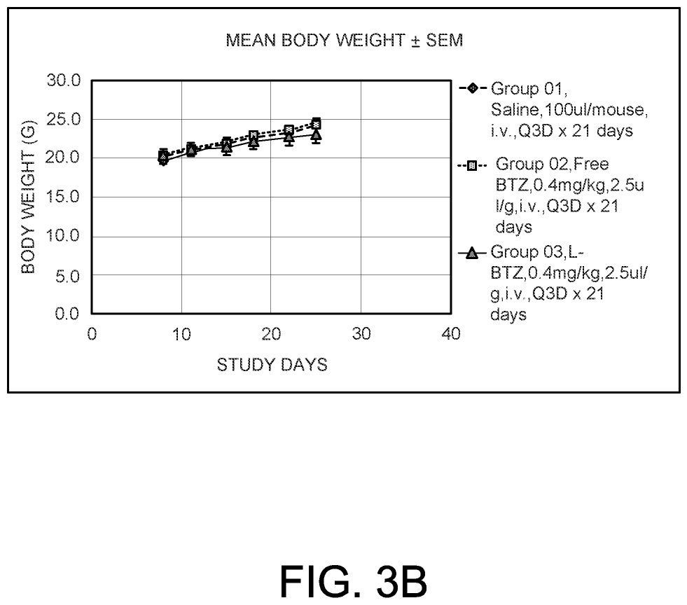

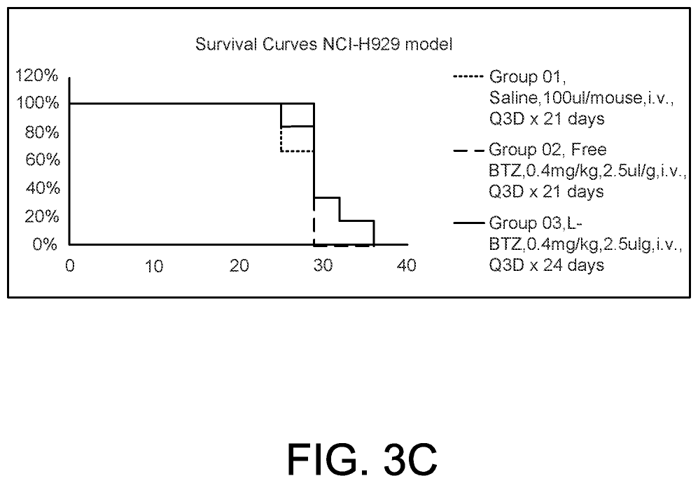

Anti-tumor effect in NCI-H929 multiple myeloma subcutaneous murine xenograft model is shown in FIG. 3A-3C. FIG. 3A shows the average tumor size of mice treated by L-BTZ was much smaller compared with mice treated by Velcade. FIG. 3B demonstrates that there is no significant body weight loss during treatment by either L-BTZ or Velcade. FIG. 3C shows there is no significant difference of survival rates between treatment group and control group during treatment.

### Plasma Pharmacokinetics of L-BTZ in Rats

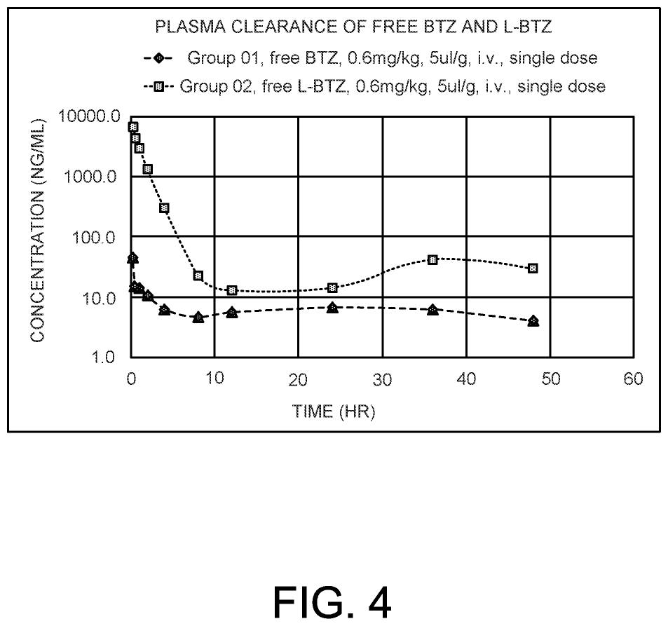

BTZ plasma clearance for L-BTZ and Velcade after single dose on rats is shown in FIG. 4. Plasma concentrations were determined by LC-MS. Compared to Velcade, the circulation time L-BTZ was much longer, which subsequently enhances the therapeutic effect and half-life time in vivo.

TABLE 1
PK parameters of free BTZ (Velcade) and L-BTZ in rats

| | Group 01, free BTZ, | Group 02, L-BTZ, |
|---|---|---|
| | 0.6 mg/kg, 5 ul/g, | 0.6 mg/kg, 5 ul/g, |
| | i.v., single dose | i.v., single dose |
| T_{1/2} (hr) | 33.1 | 6.90 |
| Tₘₐₓ(hr) | 0.25 | 0.25 |
| Cₘₐₓ(ng/ml) | 45.4 | 6600 |
| AUC₀₋ₜ (ng · hr/mL) | 322 | 10670 |
| AUC_{0-∞} (ng · hr/mL) | 864 | 18290 |
| CLss_F (mL/h/kg) | 1134 | 56 |
| Vz_F (mL/kg) | 54220 | 557 |

As shown above, L-BTZ has a much higher AUC and a lower clearance and Vd compared to BTZ. It exhibits classical PK characteristics of liposomes.

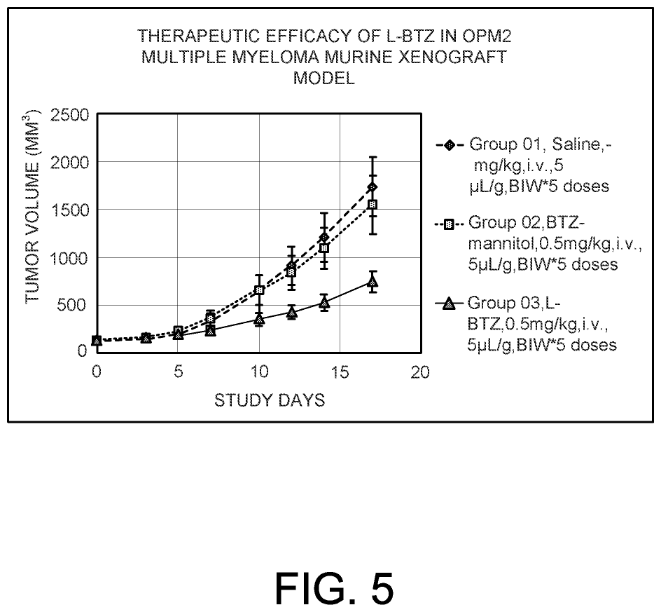

Therapeutic efficacy of L-BTZ in OPM2 multiple myeloma murine xenograft model was tested. The results are shown in FIG. 5. L-BTZ is likely to have additional therapeutic advantages over free BTZ by selectively targeting the bone-marrow niche for MM and preventing bone destruction caused by osteoclasts.

### Tiron-Based Liposomal Formulation can be Used for Delivery of Other Boronic Acid-Based Proteasome Inhibitors

High drug loading has been obtained for two other proteasome inhibitors: delanzomib and ixazomib, which have been approved for MM therapy by the FDA, using the same drug loading protocol. This was not surprising given the common boronic acid moiety in these drugs which suggest a similar ability to form stable cyclic esters with tiron in mildly basic pH. The tiron-based drug loading approach is effective for all boronic acid-based therapeutic agents and the corresponding liposomal delanzomib and liposomal ixazomib are likely to be highly effective therapeutic agents against MM and other tumors.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

1. A liposomal formulation comprising:
(i) liposomes formed from a vesicle-forming lipid; and
(ii) a capturing agent-active agent complex encapsulated in the liposomes;
wherein the capturing agent-active agent complex is a compound defined by Formula A: 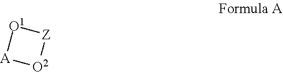
or a pharmaceutically acceptable salt thereof,
wherein:
A represents an active agent comprising a boronic acid, and
Z together with O¹ and O² represent a capture agent, wherein Z comprises an aromatic diol substituted one or more charged moieties,
wherein A is bound to the capture agent so as to form a boronic ester having the structure below

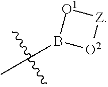

2. The liposomal formulation of claim 1, wherein the one or more charged moieties comprise one or more anionic moieties, one or more cationic moieties, or any combination thereof.

3. The liposomal formulation of claim 1, wherein the vesicle-forming lipid comprises: a lipid, a phospholipid, and a PEG-conjugated phospholipid.

4. The liposomal formulation of claim 3, wherein the lipid comprises cholesterol.

5. The liposomal formulation of claim 3, wherein the phospholipid comprises 1,2-distearoyl-sn-glycero-3-phosphocholine (DSPC),1,2-dipalmitoyl-sn-glycero-3-phosphocholine (DPPC), hydrogenated soybean phosphatidylcholine (HSPC), or sphingomyelin (SPH), distearoylphosphatidylglycerol (DSPG), dipalmitoylphosphatidylglycerol (DPPG), or dicetylphophosphate.

6. The liposomal formulation of claim 3, wherein the PEG-conjugated phospholipid comprises N-(methylpolyoxyethylene oxycarbonyl)-1,2-distearoyl-sn-glycero-3 -phosphoethanolamine (DSPE-PEG).

7. The liposomal formulation of claim 6, wherein the aromatic diol is a catechol or a furan diol.

8. The liposomal formulation of claim 7, wherein the catechol is tiron or a derivative thereof, alizarin red S or a derivative thereof, L-3,4-dihydroxyphenylalanine, D-3,4-dihydroxyphenylalanine, L,D-3,4-dihydroxyphenylalanine, or a catecholamine such as dopamine epinephrine, norepinephrine, or a derivative thereof.

9. The liposomal formulation of claim 7, wherein the furan diol is ascorbic acid or a derivative thereof.

10. The liposomal formulation of claim 1, wherein the capturing agent-active agent complex is a compound defined by Formula A-1: 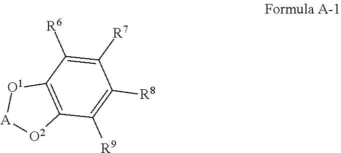
or a pharmaceutically acceptable salt thereof,
wherein:
A represents an active agent comprising a boronic acid, and
R⁶-R⁹ are independently hydrogen, oxo, hydroxy, carboxylic acid, carboxylate, sulfonic acid, sulfonate, phosphoryl group, a primary amine, a secondary amine, a tertiary amine, quaternary amine, substituted or unsubstituted alkyl group, cycloalkyl group, heterocyclyl group, aryl group, heteroaryl group, alkylaryl group, arylalkyl group, alkylcycloalkyl group, alkylheterocyclyl group, alkylheteroaryl group, alkoxy group, alkylthio group, or alkylamino group; and at least one of R⁶-R⁹ is charged moiety;
wherein A is bound to O¹ and O² so as to form a boronic ester having the structure below

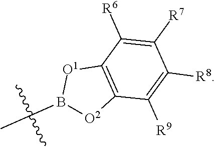

11. The liposomal formulation of claim 1, wherein the capturing agent-active agent complex is a compound defined by Formula A-2: 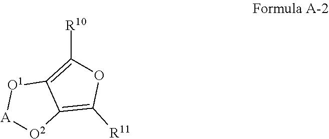
or a pharmaceutically acceptable salt thereof,
wherein:
A represents an active agent comprising a boronic acid containing active agent, and
R¹⁰ and R¹¹ are independently hydrogen, oxo, hydroxy, carboxylic acid, carboxylate, sulfonic acid, sulfonate, phosphoryl group, a primary amine, a secondary amine, a tertiary amine, quaternary amine, substituted or unsubstituted alkyl group, cycloalkyl group, heterocyclyl group, aryl group, heteroaryl group, alkylaryl group, arylalkyl group, alkylcycloalkyl group, alkylheterocyclyl group, alkylheteroaryl group, alkoxy group, alkylthio group, or alkylamino group; and at least one of R¹⁰ or R¹¹ is an charged moiety;
wherein A is bound to O¹ and O² so as to form a boronic ester having the structure below

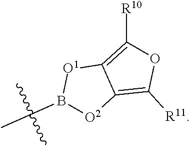

12. The liposomal formulation of claim 1, wherein the capturing agent-active agent complex is a compound defined by Formula A-3: 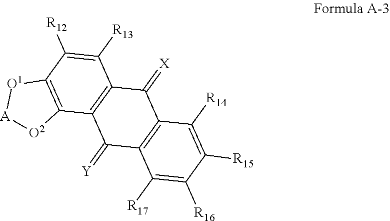
or a pharmaceutically acceptable salt thereof,
wherein:
A represents a boronic acid containing active agent, and
R¹²-R¹⁷ are independently hydrogen, oxo, hydroxy, carboxylic acid, carboxylate, sulfonic acid, sulfonate, phosphoryl group, a primary amine, a secondary amine, a tertiary amine, quaternary amine, substituted or unsubstituted alkyl group, cycloalkyl group, heterocyclyl group, aryl group, heteroaryl group, alkylaryl group, arylalkyl group, alkylcycloalkyl group, alkylheterocyclyl group, alkylheteroaryl group, alkoxy group, alkylthio group, or alkylamino group; and at least one of R¹²-R¹⁷ is a charged moiety;
wherein A is bound to O¹ and O² so as to form a boronic ester having the structure below

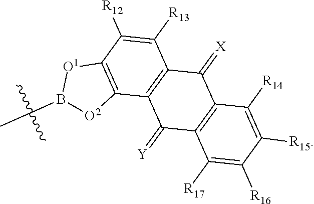

13. The liposomal formulation of claim 1, wherein the boronic acid containing active agent comprises a boronic acid containing anticancer agent, a boronic acid containing antimicrobial agent, a boronic acid containing antiproliferative agent, a boronic acid containing antibiotic agent, a boronic acid containing antimitotic agent, a boronic acid containing antiviral agent, a containing boronic acid prodrug (e.g., boronic acid containing camptothecin prodrug, boronic acid containing methotrexate prodrug, or boronic acid containing crizotinib prodrug), a boronic acid containing proteasome inhibitor, a boronic acid containing autotaxin inhibitor, an urea-containing peptide boronic acid, a boronic acid containing histone deacetylases inhibitor, a boronic acid β-lactamase inhibitor, a boronic acid containing sensor (e.g., boronic acid containing photo induced electron transfer materials), a poly(aniline boronic acid) polymers, a boronic acid containing carbohydrate sensor, a boronic acid containing dopamine sensor, a boronic acid containing cholesterol analog, a boronic acid containing chalcone, a boronic acid containing peptide.

14. The liposomal formulation of claim 1, wherein the capturing agent-active agent complex is: 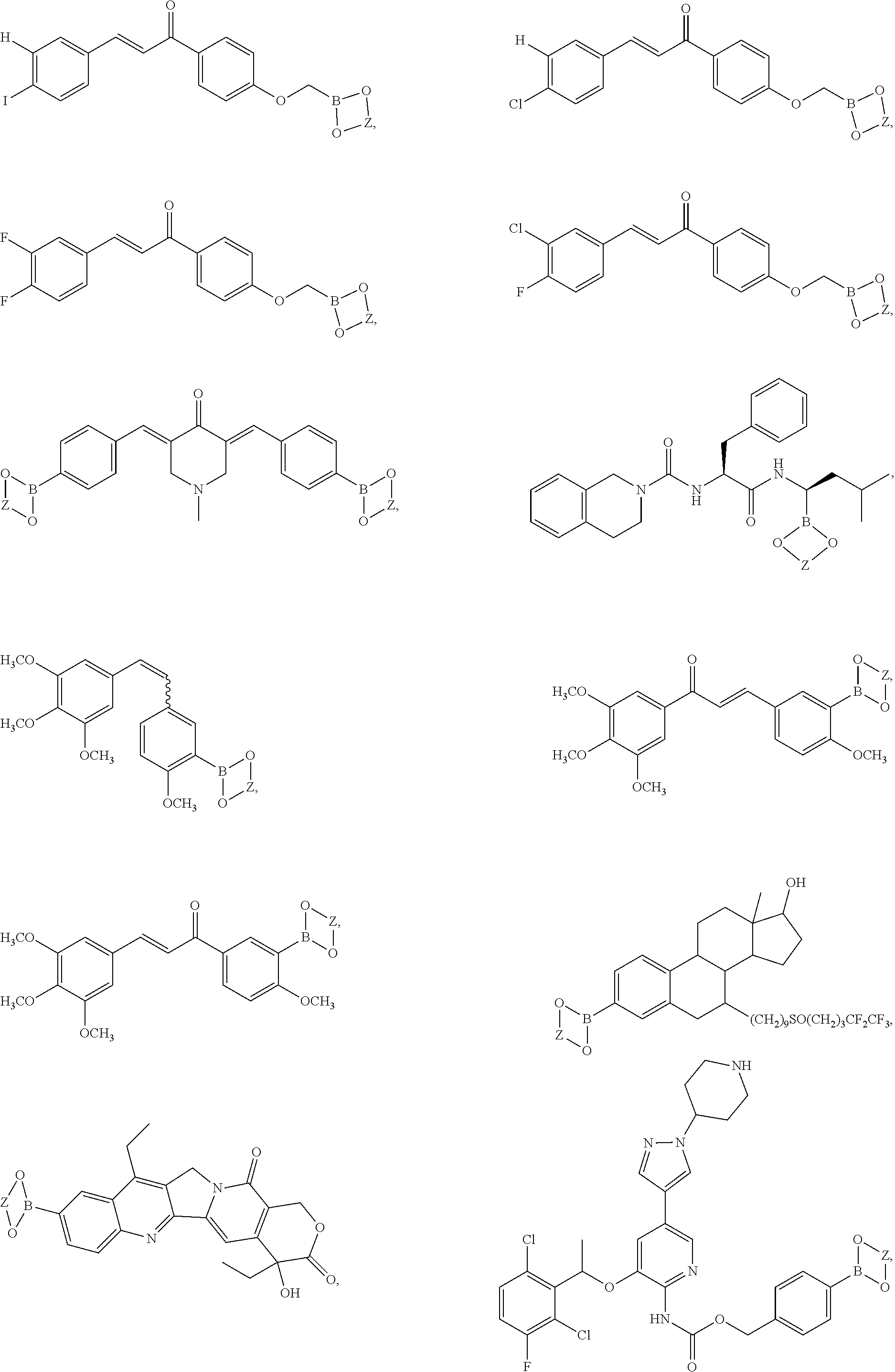 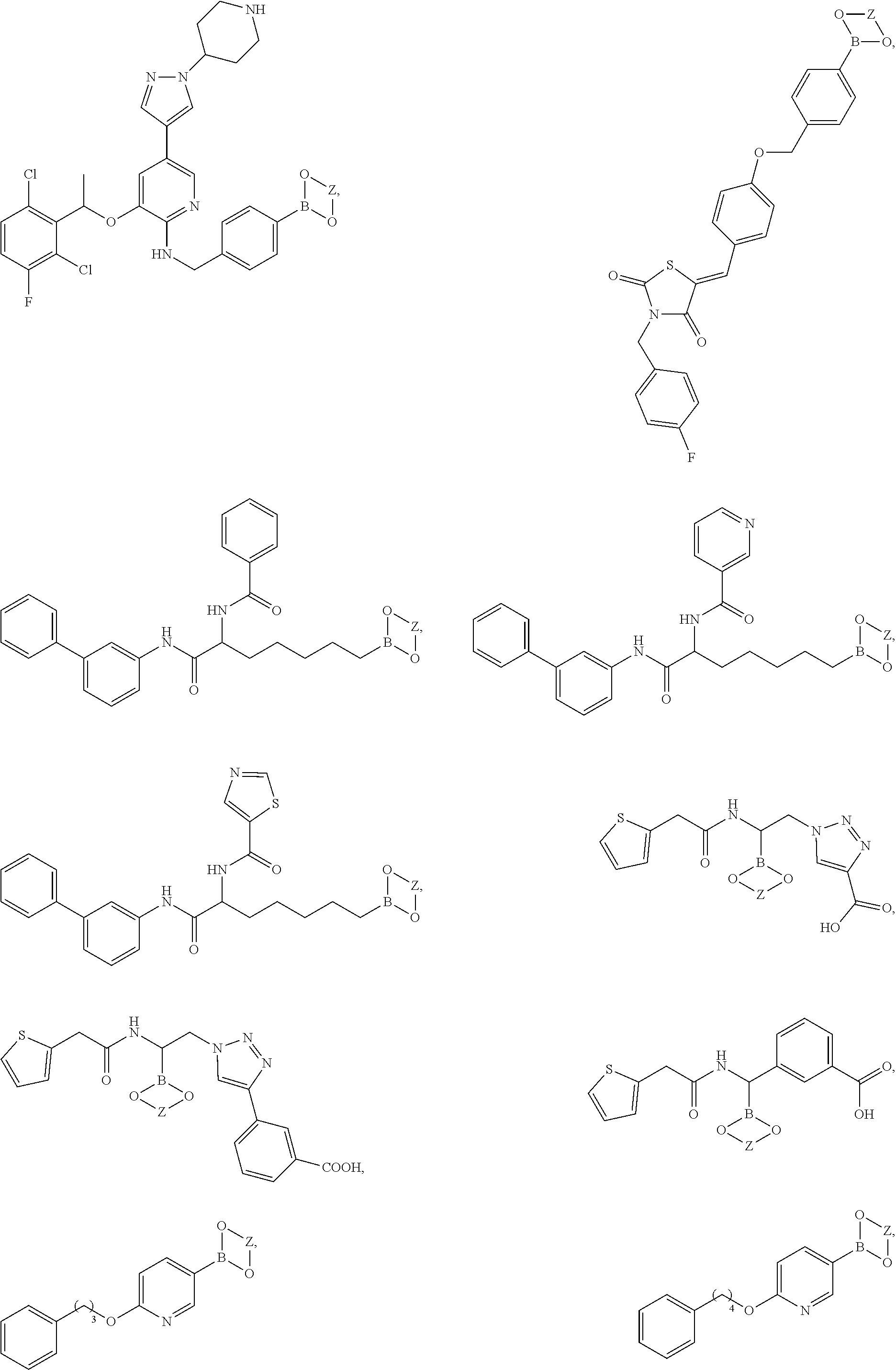 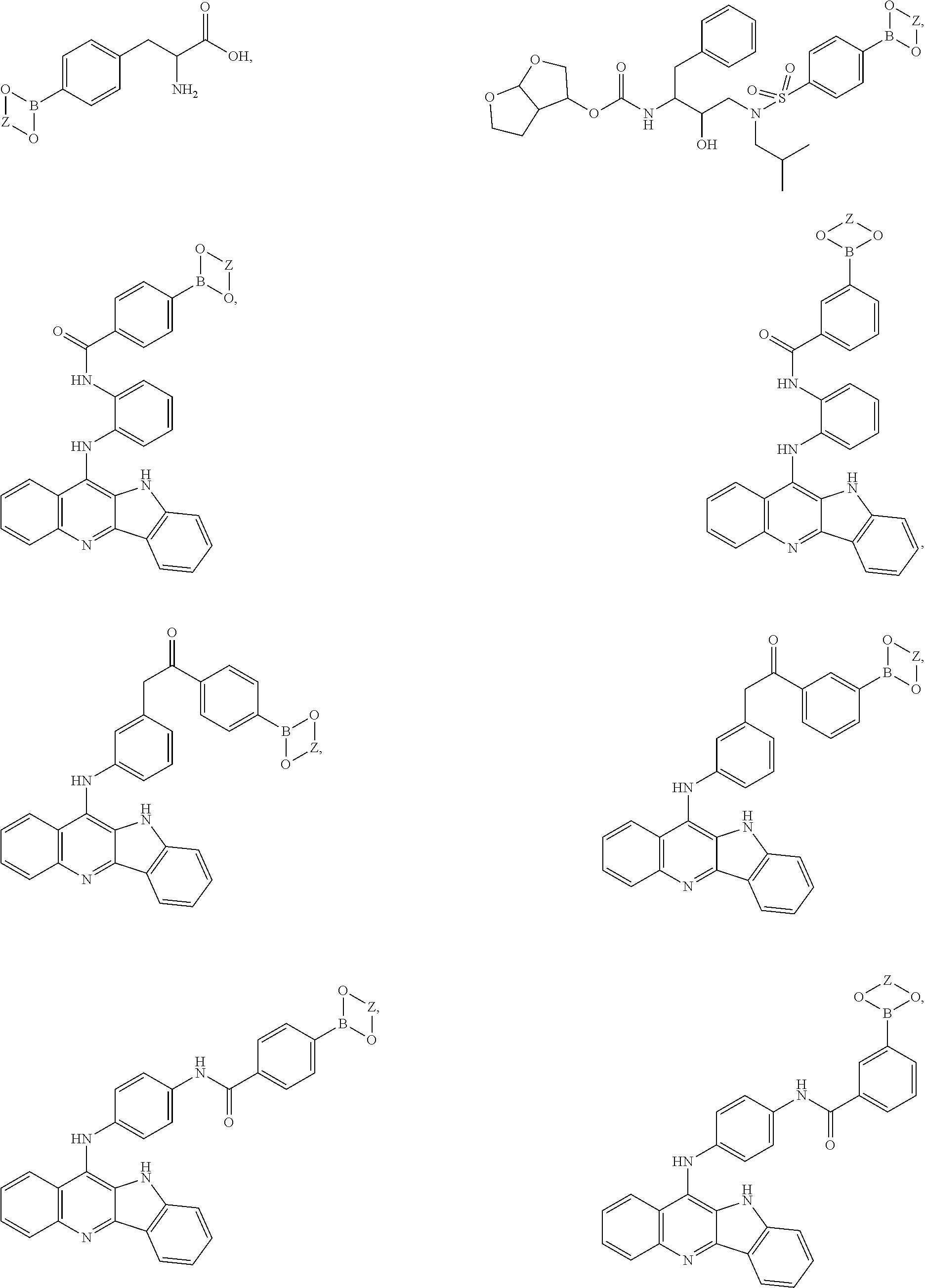 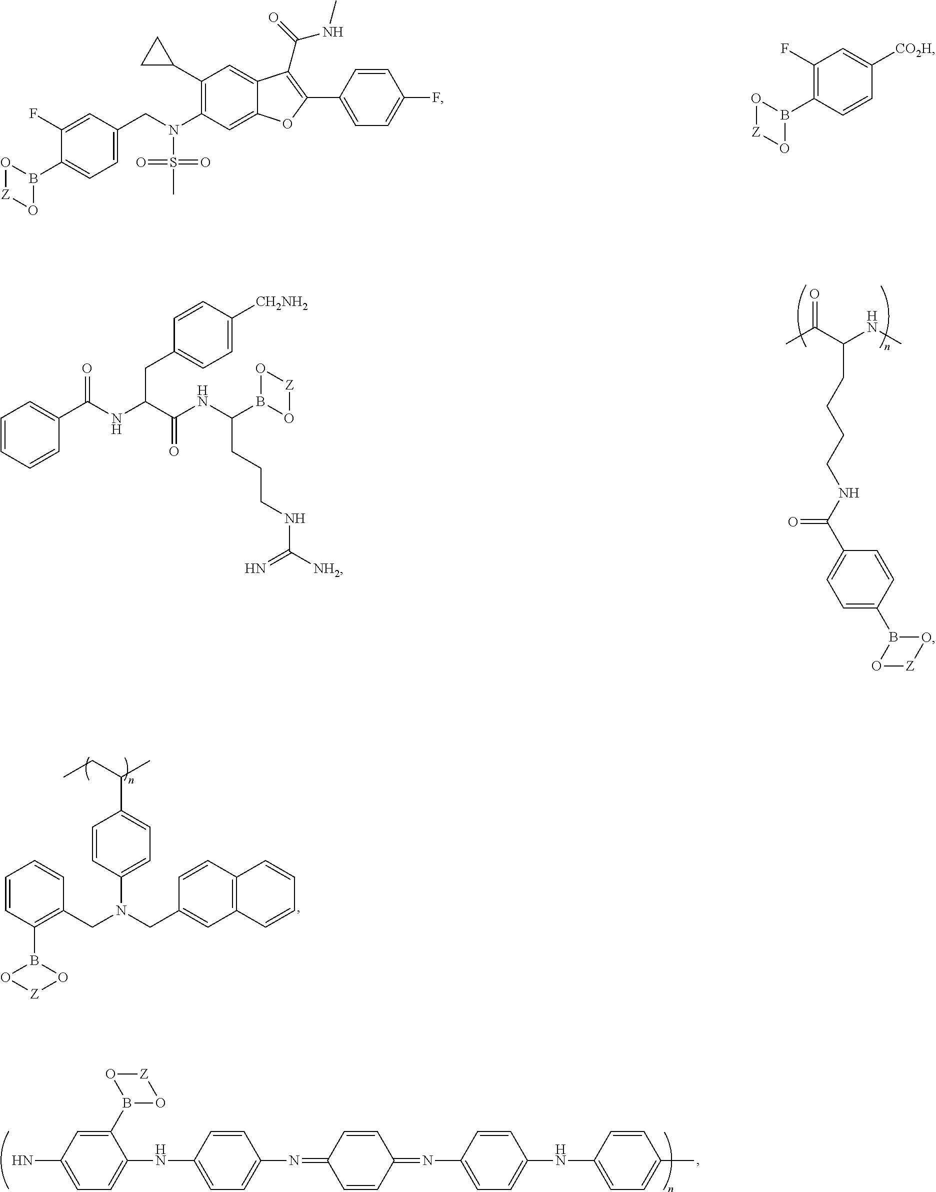 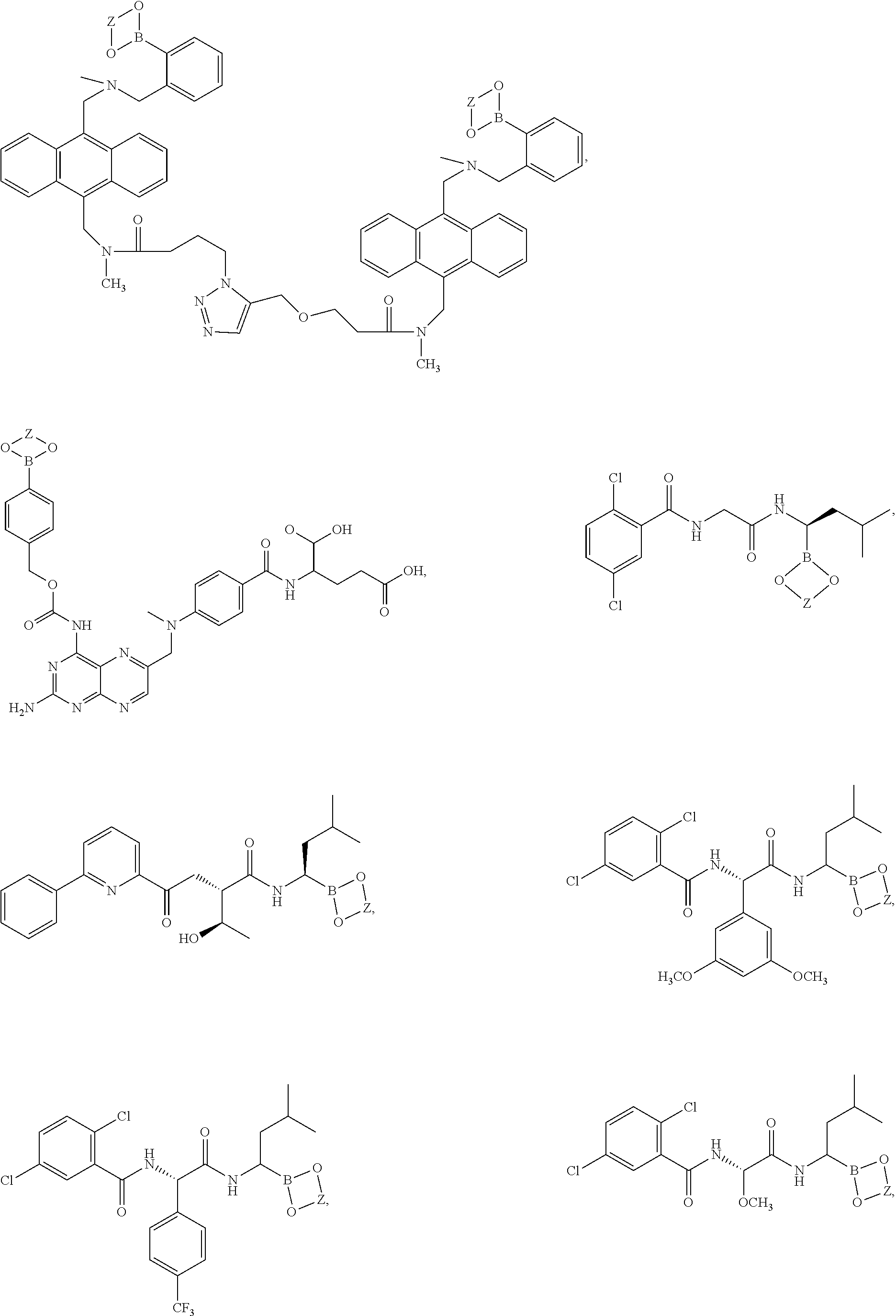 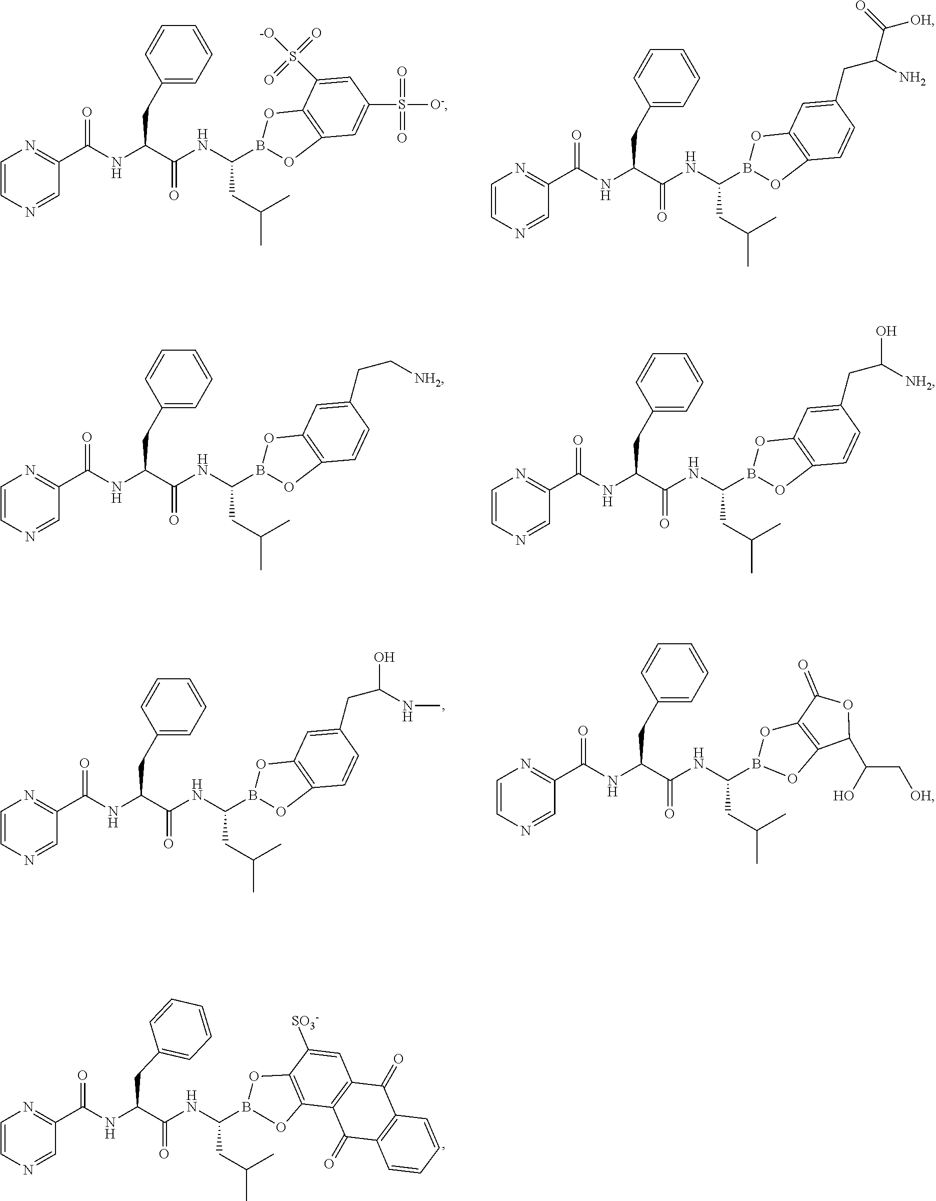
or a pharmaceutically acceptable salt thereof.

15. A liposomal formulation comprising:
(i) liposomes formed from a vesicle-forming lipid; and
(ii) a capturing agent-active agent complex encapsulated in the liposomes;
wherein the capturing agent-active agent complex is a compound defined by Formula I: 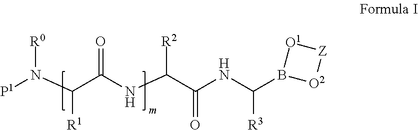
wherein
P¹ is hydrogen or an amino-group protecting moiety;
R⁰ is hydrogen or an alkyl group;
R¹, R², and R³ are independently hydrogen, an alkyl group, a cycloalkyl group, a heterocyclyl group, an aryl group, a heteroaryl group, an alkoxy, or —CH₂—R⁴;
R⁴ is an aryl group, an alkylaryl group, an arylalkyl group, a cycloalkyl group, an alkylcycloalkyl group, a heterocyclyl group, an alkylheterocyclyl group, a heteroaryl group, an alkylheteroaryl group, an alkoxy group, or an alkylthio group;
m is 0, 1, or 2; and
Z, together with O¹ and O², represent a capture agent, wherein Z comprises one or more charged moieties;
or a pharmaceutically acceptable salt thereof.

16. The liposomal formulation of claim 15, wherein the one or more charged moieties comprise one or more anionic moieties, one or more cationic moieties, or any combination thereof.

17. The liposomal formulation of claim 15, wherein the vesicle-forming lipid comprises: a lipid, a phospholipid, and a PEG-conjugated phospholipid.

18. The liposomal formulation of claim 17, wherein the lipid comprises cholesterol.

19-52. (canceled)

53. A method of treating a cancer in a subject in need thereof, comprising administering a therapeutically effective amount of a formulation defined by claim 1.

54. (canceled)
